(19) United States
(12) Patent Application Publication
(10) Pub. No.: US 2004/0247555 A1
(43) Pub. Date: Dec. 9, 2004

(54) Methods of and compositions for modulating hair growth via P-cadherin modulators

(76) Inventors: Eli Sprecher, Tivon (IL); Reuven Bergman, Haifa (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/678,160

(22) Filed: Oct. 6, 2003

Related U.S. Application Data
(60) Provisional application No. 60/418,163, filed on Oct. 15, 2002.

(51) Int. Cl. A61K 48/00; A61K 7/06; A61K 7/11; A61K 39/395

(52) U.S. Cl. 424/70.14; 424/70.13; 424/146.1; 514/44

(57) ABSTRACT

A method of identifying a hair growth modulator (i.e., hair growth inhibitor or inducer) which comprises identifying a P-cadherin modulator (i.e., P-cadherin inhibitor or inducer); and testing whether the P-cadherin modulator is functional as a hair growth modulator.

This application claims the benefit of priority from U.S. provisional patent application No. 60/418,163, filed Oct. 15, 2002

### FIELD AND BACKGROUND OF THE INVENTION

The present invention relates to methods and pharmaceutical compositions for modulating hair growth, and, more particularly, to methods and pharmaceutical compositions for inducing hair growth in cases of alopecia and methods and pharmaceutical compositions for inhibiting hair growth at locations where hair is unwanted, using modulators of P-cadherin.

Alopecia (baldness) is a deficiency of hair, either normal or abnormal, and is primarily a cosmetic problem in humans, although the negative psychological impact of hair loss is well known. See C. H. Mortimer et al., Clin. Exp. Dermatol. 9, 342-350 (1984). Dermatologists recognize many different types of alopecia, with androgenic alopecia being the most common cause of hair loss in both men and women. As this type of hair loss is more common and more severe in males, it is typically referred to as “male pattern baldness”. However, it is thought that androgenic alopecia affects more that one third of individuals of either sex who have a strong family history of hair loss. See W. F. Bergfield, Clin. Dermatol. 6, 102-107 (1988).

One traditional treatment for alopecia is the method of hair transplantation. Typically, this method involves transplanting plugs of natural hair from areas of the scalp where hair is growing to bald areas. This procedure is costly, time-consuming, painful, and meets with only limited success.

Another common treatment for hair loss is the application of a chemical or drug for the purpose of stimulating hair growth. For example, U.S. Pat. No. 5,177,061 to Pickart proposes the topical application of glycyl-L-histidyl-L-lycine:copper(II) (GHL-Cu) and its derivatives to promote hair growth in warm-blooded animals. U.S. Pat. No. 4,832,946 to Green proposes a composition for topical application to mammalian hair or skin, comprising an amount of the cell-free supernatant from a culture of dermal papilla fibroblasts, which is said to increase hair growth in the rat. U.S. Pat. No. 5,358,714 to Green proposes the use of diacylglycerol activators of protein kinase C in order to increase or maintain hair growth in mammals, while U.S. Pat. No. 5,068,315 to Buultjens et al. proposes the application of purified hair growth regulating peptides (HGRP) to stimulate hair growth. It has also been suggested that retinoids, substituted pyrimidines, and immunosuppressants be used as possible treatments for hair loss, although methods utilizing these compounds have not been entirely successful in producing a reliable and safe method of inducing hair growth. See G. Bazzano et al., J. Invest. Dermatol. 101 (1 Supplement), 138S-142S (1993), H. Jiang et al., J. Invest. Dermatol. 104(4), 523-525 (1995).

In recent years, the topical application of minoxidil has been a widely-used method for treating androgenic alopecia. See A. R. Zapacosta, N. Eng. J. Med. 303, 1480-81 (1980). U.S. Pat. No. 4,139,619 to Chidsey, proposes a topical composition of minoxidil and related iminopyrimidines to stimulate the conversion of vellus hair to terminal hair and increase the rate of growth of terminal hair. However, despite its popularity, minoxidil has not performed in a completely satisfactory fashion in promoting hair growth in all target populations.

The following provides further insight with respect to pharmaceuticals used with limited success to treat alopecia.

Thymosin fraction 5 (TF5) is a partially purified mixture of polypeptides prepared from calf thymus glands. TF5 has been routinely prepared from calf thymus. However, it may also be prepared from porcine, ovine, murine, goat, rat, chicken, and human thymus tissues. Preparation and isolation of TF5 have been described (Hooper et al., “The purification and properties of bovine thymosin”, Ann. NY Acad. Sci. 249:125, 1975). TF5 consists of at least 40 to 50 distinct polypeptides on isoelectric focusing on polyacrylamide gel plates (pH 3.5-9.5). TF5 is essentially free of lipids, carbohydrates and endotoxins. TF5 has been demonstrated to be effective in reconstituting immune functions in thymic-deprived or immunodeprived animals, in humans with primary immunodeficiencies, and in immunosuppressed cancer patients. A primary effect of this mixture of peptides is to stimulate cell-mediated immunity. Two of the major biologically active ingredients in TF5 are thymosin alpha1 (Talpha1) an immunomodulatory peptide of 28 amino acids (molecular weight 3,108 daltons) (Low et al., “The chemistry and biology of Thymosin I. Isolation and characterization and biological activities of Tα₁ and polypeptide beta1 from calf thymus,” J. Bio. Chem. 254:981, 1979), and thymosin β4 (Tβ₄), an actin-sequestering peptide of 43 amino acids (molecular weight 4,963 daltons) (Low, T. L. K., and Goldstein, A. L., “Chemical characterization of thymosin 4,” J. Bio. Chem. 257:1000, 1982). Tα₁ and Tα₄ are highly conserved in nature and their amino acid sequences are identical in most mammalian species. More than a dozen TF5-like preparations have been prepared from calf or porcine thymus tissue. These thymic extracts such as thymostimulin (TP-1), TFX, thymalin, thymoject, thym-Uvocal, and others, are variations of the TF5 formulation and are all partially purified preparations composed primarily of polypeptide mixtures with molecular weights of 15,000 or less. The major biologically active components of TF5 contain Tα₁ and Tα₄, as well as lower concentrations of other purified well characterized thymosin peptides such as prothymosin a (Pro Tα₁), Tα₂ to Tα₁ and Tβ₃, Tβ to Tβ₁₃, MB3S, MB40, ubiquitin, thymulin (FTS), thymic humoral factor (THFα₂) and thymopoietin (TP). The TF5-like extracts prepared by variations of the procedure used originally to prepare TF5 may also contain alpha and beta as key ingredients and smaller quantities of the other peptides described in TF5 such as Pro Tα₃, FTS, THFα₂, TP, ubiquitin and MB 35 and MB 40. Thymosin fraction 5 was found useful in the treatment of alopecia.

Substances that block DHT, testosterone, estradiol and EGF are thus believed to be of value in the prevention and treatment of alopecia. Systemic antiestrogens that have been used include tamoxiten citrate, a variety of triphenylethylene-based compounds and testolaotone.

Various azoles, especially ketoconazole have been found to have a significant role in the treatment of alopecia. Ketoconazole is important because it also blocks testosterone, DHT, and estrudiol non-specifically. However, systemic treatment to this compound over a long period of time results in loss of libido in men and women. In the context of topical treatment, this problem does not occur, and the effect relative to alopecia is much more significant. Undecylenic acid and a variety of systemic preparations may also be employed. These include grisocfulvia, terbinafine and fluconazole and other azoles, as well as ampotercin B and ampotercin like compounds.

Surprisingly, bioflavanoids can inhibit the production of epidermal growth factor (EGF). The most powerful of these, quercetin methyl chalcone, is water soluble. This compound effectively blocks EGF in relatively low concentrations. This greatly reduces hair loss and contributes significantly to hair growth. Polyamines also have this ability. Putrescine, protamine, etc., all will promote hair regrowth by blocking EGF. However, these substances are not cosmetically preferable for topical use because of their odor. It has been found that compounds containing bioflavanoids, especially quercetin methyl chalcone, greatly reduce hair loss and facilitate hair regrowth.

The presence of an ectoparasite and its role in alopecia prompted the development of an effective mitocide. Using fragrance-based chemicals, a skin penetrant, preferably PX-13, and a surfactant, it was discovered that this parasite could be effectively eliminated. Concomitantly, it was discovered that this composition was capable of effectively killing any mite, insect or chitin-coated organism. This was completely unexpected. Although others have recognized the efficacy of fragrance moieties in an aerosolized format, the novelty represented by this invention is inherent in the concomitant administration of a surfactant and an antilipase composition (such as PX-13, U.S. Pat. No. 5,659,055).

Certain indole-based compounds have a significant effect on hair loss. These include but are not limited to indole, skatole, indole-3-carbinol, and melatonin. They exert their effect by blocking the effects of virtually all estrogens. Melatonin has been used in high doses orally as an effective birth control agent, and a combination of indole-3-carbinol and melatonin is more powerful than either alone. Further, these compounds have antifungal properties. It should also be noted that very high concentrations of indole are found in jasmine fragrance and citrus flower based fragrances such as orange and lemon.

Melatonin has been found to alter the cyclic pattern of hair growth in rodents. Melatonin compositions and methods of using these melatonin compositions have been developed for treating the cosmetic and physical appearance of the scalp. (Pierpaoli, W., Regelson, W., Melatonin Compositions and Uses Thereof. U.S. Pat. No. 4,746,674 (1988)).

Melatonin was found to increase the 5-α reductase of seminiferous tubules for both progesterone and testosterone. Melatonin decreased androgen synthesis in both testicular interstitial cells and tubules. Currently, 5-α reductase modulating agents are being used to treat male pattern baldness.

Melatonin inhibits estrogen-mediated cell proliferation in MCF-7 cancer cells (Cos, S. Blask, D. E., Melatonin Modulates Growth Factor Activity in MCF-7 Human Breast Cancer Cells. J. Pineal Research 17:25-32 (1994). It was shown that melatonin down-regulates estrogen receptor expression. This group also showed that messenger RNA (MRNA) estrogen-receptor-mediated expression is inhibited by melatonin in MCF-7 breast cancer cells (Molis, T. M., Spriggs, L. L. Hill, S. M., Modulation of Estrogen Receptor mRNA Expression by Melatonin in MCF-7 Human Breast Cancer Cells. Mol. Endocrinol. 8: 1681-90 (1994).

The inhibitory mechanism of melatonin relates to effects on cell cycle response resulting from a block to estrogenic growth stimulation, perhaps through effects on estrogen receptor availability.

Although a variety of treatments are presently offered to treat alopecia, not all subjects are responsive to such treatments, whereas some treatments are associated with unwanted side effects

Hence, there is still a great need for an efficient treatment for alopecia, which will overcome the limitations of the presently employed treatments and will offer an alternative to at least a subset of the patients.

While alopecia affects some individuals, other individuals suffer excessive hair growth and/or are culturally influenced by the trend of hairless body and hence treatments for the removal of hair are at their highest demand. Various methods of hair removal are known. For example, the hair can be shaved from the body or can be removed by the use of tweezers or other instruments which pluck the hairs from the skin, such as devices including bent rotating coil springs and the like. In addition, chemical depilatory preparations and waxes have been formulated for the purpose of hair removal. Conventional depilatory preparations, often containing sulphide chemicals, act by weakening the structure of the hair to such an extent that scraping the cream off the skin breaks the hair at skin level and thus removes it. Alternatively, waxes can be applied to the skin which can then be peeled away with the hairs embedded therein.

Each of these methods has attendant disadvantages. Shaving brings only temporary alleviation since the roots of the hair are still present and the hair will grow again after a very short period. Also, there is the danger of cutting the skin on shaving. Chemical depilatory preparations tend to have an unpleasant smell and the use of waxes and coil spring devices can cause some discomfort.

Currently, the most common methods for hair removal involve the use of air removal creams, as well as shaving, waxing and electrolysis. Although reams and shaving are popular because they can be readily used at home, they are inadequate because they must be used on a regular basis. Waxing and electrolysis offer longer term hair removal. Both methods, however, can be time-consuming and are often quite painful. For example, removing a typical mustache which contains 1,000 to 2,000 hairs by electrolysis may take up to 50 visits before the hair removal is complete.

More recently, lasers alone or in conjunction with topical formulations containing carbon particles, hair dyes, hematoporphyrin derivatives or aminolevulinic acid have been used for hair removal (See, U.S. Pat. Nos. 5,226,907 and 5,425,728; Grossman, M. et al. Lasers Surg. Med. Suppl. 7:44 (1995)). Such treatments are generally not selective in that they result in only partial destruction of hair follicles and may promote skin reaction.

All of these hair removal treatments fail to prevent new hair growth. Hirsutism is defined as terminal hair growth in women in a pattern typical of men. Current modalities include the use of cosmetic means, anti-androgen therapy such as oral contraceptives, cyproterone aceate, spironolactone with moderate success rate and many associated side effects.

Accordingly, there exists a great need for an efficient method of inhibiting hair growth.

The present invention emerges from a novel discovery that a mutation in the CDH3 gene which encodes P-cadherin is the cause for the autosomal recessive disorder congenital hypotrichosis which is associated with juvenile macular dystrophy (HJMD; MIM601553), and is characterized by hair loss heralding progressive macular degeneration and early blindness (Souied, E. et al. Ophthalmic Genet. 16, 11-15 (1995); Raison-Peyron, N. et al. Br. J. Dermatol. 143, 902-904 (2000); Da Cruz, L. & McAllister, I. L. Br. J. Ophthalmol. 85, 239 (2001)).

Using homozygosity mapping in 4 consanguineous families, the HJMD gene was localized to 16q22.1. This region harbors CDH3 encoding P-cadherin, which is expressed in the retinal pigment epithelium and hair follicles. Mutation analysis revealed in all families revealed a common homozygous deletion in exon 8 of CDH3. These results establish the molecular etiology of HJMD and implicate for the first time a cadherin molecule in the pathogenesis of a human hair and retinal disorder.

### SUMMARY OF THE INVENTION

According to one aspect of the present invention there is provided a method of identifying a hair growth modulator (i.e., hair growth inhibitor or inducer) comprising identifying a P-cadherin modulator (i.e., P-cadherin inhibitor or inducer); and testing whether the P-cadherin modulator is functional as a hair growth modulator.

According to another aspect of the present invention there is provided a method of identifying a hair growth modulator comprising identifying a molecule being capable of specifically binding to P-cadherin; and testing whether the molecule is functional as a hair growth modulator.

According to yet another aspect of the present invention there is provided a method of modulating (i.e., inhibiting or inducing) hair growth, the method comprising administering to a subject in need a therapeutically effective amount of a P-cadherin modulator (i.e., P-cadherin inhibitor or inducer) functional as a hair growth modulator.

According to still another aspect of the present invention there is provided a pharmaceutical composition for modulating hair growth, the pharmaceutical composition comprising, as an active ingredient, a therapeutically effective amount of a P-cadherin modulator functional as a hair growth modulator.

According to further features in preferred embodiments of the invention described below, the pharmaceutical composition further comprising, as an additional active ingredient, a therapeutically effective amount of an additional hair growth modulator (i.e., an additional hair growth inhibitor or inducer, respectively).

According to still further features in the described preferred embodiments, the P-cadherin modulator is an antisense oligonucleotide capable of specifically binding to P-cadherin gene, pre-messenger RNA or messenger RNA under physiological conditions and hence serves as a P-cadherin inhibitor.

According to still further features in the described preferred embodiments the P-cadherin modulator is an antisense construct encoding an antisense transcript capable of specifically binding to P-cadherin gene, pre-messenger RNA or messenger RNA under physiological conditions.

According to still further features in the described preferred embodiments the P-cadherin modulator is a polynucleotide capable of directing P-cadherin expression in hair follicle cells and hence serves as a P-cadherin inducer.

According to still further features in the described preferred embodiments the P-cadherin modulator or the molecule capable of binding P-cadherin is an anti-P-cadherin antibody and hence serves as a P-cadherin inhibitor.

According to still further features in the described preferred embodiments the P-cadherin modulator or the molecule capable of binding P-cadherin is an a small molecular weight organic compound, which may serve as either a P-cadherin inhibitor or inducer.

According to still further features in the described preferred embodiments identifying the molecule being capable of specifically binding to P-cadherin is by a two hybrid system.

According to an additional aspect of the present invention there is provided a hair growth modulator identified by the method described herein.

According to yet an additional aspect of the present invention there is provided a method of modulating hair growth comprising administering to a subject in need a therapeutically effective amount of the hair growth modulator described herein.

The present invention successfully addresses the shortcomings of the presently known configurations by providing new means with which to modulate hair growth.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-e demonstrate clinical spectrum of HJMD. 1a, Sparse, short hair on the scalp of a 17-year old affected individual; 1b, Scanning electron microscopy of a hair shaft. Note the fusiform beading along the hair shaft (original magnification X70), reminiscent of pseudomonilethrix (MIM177750). This abnormality is due to flattening of the shaft (arrow) seen in details in insert (original magnification X 500); 1c, Pili torti (180° twisting of the hair) apparent by light microscopy (original magnification X100); 1d, Eye fundus examination in HJMD. Note atrophic scars of the macular area surrounded by degenerative pigmentary changes; 1e, Electroretinogram of a HJMD patient (left) compared to a normal profile (right) demonstrating reduced wave amplitude, consistent with macular dysfunction.

FIGS. 2a-g demonstrates a mutation in CDH3 which underlies HJMD. 2a, Haplotype analysis in 4 HJMD families using 6 polymorphic markers on 16q22.1. The shared disease-associated haplotype is boxed; 2b, Sequence analysis reveals a homozygous G deletion at cDNA position 981 of CDH3 in patient 22 (left panel); each parent carries this mutation in a heterozygous state (middle panel); the wildtype (WT) sequence is shown in the right panel; 2c, Segregation of the 981delG in family 1 is illustrated by restriction fragment analysis. 981delG causes loss of an enzyme recognition site for NlaIII. Upon digestion, amplicons of exon 8 of CDH3 (320 bp), normally resulting in three fragments (individuals 8 and 19), yields only two fragments in affected individuals (3 and 9) and four fragments in heterozygous carriers of the mutation (individuals 5 and 1); 2d, Predicted wildtype (black) and mutant (red) amino acid sequence of P-cadherin; 2e, Expression of CDH3 in the skin of a patient (P) and a control (C) determined by RT-PCR amplification of RNA using gene-specific intron-crossing primers for CDH3 and β-actin; 2f, Schematic representation of the wildtype and predicted mutant protein structures; 2g, Immunostaining of fresh frozen skin biopsies obtained from a patient and a control with antibodies specific for P-cadherin (P-cad) or E-cadherin (E-cad) (Santa Cruz) (original magnification X 630). E-cadherin is expressed both in control and patient skin. Note reduced staining for P-cadherin in the patient epidermis (left upper panel) and follicular epithelium (right upper panel).

FIGS. 3a-p show multiple alignment of human cadherin cDNAs.

Multiple alignment was made using ‘clustalW’ software (from EMBL) with all parameters set on default. Bases common to all cadherins are marked with an asterisks.

FIGS. 4a-d show multiple alignment of human cadherin cDNAs.

Multiple alignment was made using ‘clustalW’ software (from EMBL) with all parameters set on default. For each precursor protein the first 21 amino acids from the N′ serve as signal peptide. The bold and underlined letters in each sequence represent the transmembrane domain. The sequence up-stream to the trans membrane domain is the extracellular. The sequence down stream is the cytoplasmic part of the protein. Perfect alignment between cadherin family members is marked at the bottom of every cluster. In order to select for immunogenic peptides of P-cadherin regions of low similarity were analyzed for immugenicity using the ‘peptidestructure’ software of the ‘GCG package’.

### DESCRIPTION OF THE PREFERRED EMBODIMENTS

The present invention is of methods and pharmaceutical compositions which can be used to modulate hair growth. Specifically, the present invention can be used to (i) treat alopecia (boldness) or otherwise induce hair growth on the one hand; and to (ii) inhibit hair growth, in cases of excessive hairiness or for cosmetic purposes, on the other hand. The invention is further of methods of identifying P-cadherin modulators effective in either inducing hair growth in cases of alopecia and inhibiting hair growth in cases of excessive hairiness and/or for cosmetic reasons.

The principles and operation of methods and pharmaceutical composition according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Although P-cadherin was originally identified more than 10 years ago and was shown to be expressed in the mouse hair follicles, nothing was known until recently about its role in the morphogenesis of the hair follicle. The inventors of the present invention identified 4 families affected with congenital hypotrichosis associated with juvenile macular dystrophy (HJMD; MIM601553). Juvenile macular dystrophy is an autosomal recessive disorder of unknown etiology characterized by hair loss heralding progressive macular degeneration and early blindness (Souied, E. et al. Ophthalmic Genet. 16, 11-15 (1995); Raison-Peyron, N. et al. Br. J. Dermatol. 143, 902-904 (2000); Da Cruz, L. & McAllister, I. L. Br. J. Ophthalmol. 85, 239 (2001)). Using homozygosity mapping in these consanguineous families, the HJMD gene was localized to chromosome 16q22.1. This region harbors the CDH3 gene encoding P-cadherin, which is expressed in the retinal pigment epithelium and hair follicles. Mutation analysis revealed in all families a common homozygous deletion in exon 8 of CDH3. These results establish the molecular etiology of HJMD and positively demonstrate for the first time the importance of P-cadherin in the morphogenesis of the hair follicle. These findings pave the way for various novel therapeutic strategies based on the modulation of P-cadherin in hair disorders such as the design of P-cadherin inhibitors for the treatment of unwanted hair growth, such as hirsutism.

Given the fact that P-cadherin is necessary for the morphogenesis of the hair follicle; and given the fact that lack of functional P-cadherin is not associated with any skin phenotype, it is clear that modulation of P-cadherin function represents an attractive strategy for modulating hair growth in for example hirsutism or for cosmetic reasons.

Hirsutism is defined as terminal hair growth in women in a pattern typical of men. Current modalities include the use of cosmetic means, anti-androgen therapy such as oral contraceptives, cyproterone aceate, spironolactone with moderate success rate and many associated side effects. The design of such inhibitors may be based on the use of specific antisense oligonucleotides transferred using novel and efficient methods targeted to the hair follicle Domashenko et al, Nature Biotechnol 18, 43-47 (2000), which is incorporated herein by reference). Such a strategy has been successful with another regulator of hair growth, the hairless protein, in a murine model (Cserhalmi-Friedman, P. B. & Christiano, A. M. J Invest Dermatol, in press, and incorporated by reference herein). Alternatively, the well-known structure of P-cadherin may be amenable to computer-based inhibitor designing.

On the other hand, correction or partial correction of hair loss in HJMD and other alopecia patients may be achieved by the use of a P-cadherin inducer. Partial correction of hair loss in HJMD patients during puberty indicates that P-cadherin expression is involved in the androgen-mediated regulation of hair growth. Indeed, expression of several cadherins have been shown to be controlled by sex hormones.

Hence, according to one aspect of the present invention there is provided a method of identifying a hair growth modulator (i.e., hair growth inhibitor or inducer). The method according to this aspect of the present invention is materialized by identifying a P-cadherin modulator (i.e., P-cadherin inhibitor or inducer); and thereafter testing whether the P-cadherin modulator is functional as a hair growth modulator.

According to another aspect of the present invention there is provided a method of identifying a hair growth modulator. The method according to this aspect of the present invention is materialized by identifying a molecule capable of specifically binding to P-cadherin; and thereafter testing whether the molecule is functional as a hair growth modulator.

According to yet another aspect of the present invention there is provided a method of modulating (i.e., inhibiting or inducing) hair growth. The method according to this aspect of the present invention is materialized by administering to a subject in need a therapeutically effective amount of a P-cadherin modulator (i.e., P-cadherin inhibitor or inducer) functional as a hair growth modulator.

According to still another aspect of the present invention there is provided a pharmaceutical composition for modulating hair growth. The pharmaceutical composition comprising, as an active ingredient, a therapeutically effective amount of a P-cadherin modulator functional as a hair growth modulator.

Preferably, the pharmaceutical composition further comprises, as an additional active ingredient, a therapeutically effective amount of an additional hair growth modulator (i.e., an additional hair growth inhibitor or inducer, respectively). Such hair growth modulators (both hair growth inhibitors and hair growth inducers) are discussed at length at the Background section and elsewhere hereinabove.

As used herein, the phrase “P-cadherin modulator” includes any and all molecules capable of increasing or decreasing specifically P-cadherin expression and/or P-cadherin function, such as binding β-catenin and/or other cellular skeleton components.

As used herein the term “specifically” refers to an effect which is unique to P-cadherin expression of activity and not to other cadherins or other cell components.

As used herein, the phrase “P-cadherin inhibitor” includes any and all molecules capable of decreasing specifically P-cadherin expression and/or P-cadherin function, such as binding β-catenin and/or other cellular skeleton components.

As used herein, the phrase “P-cadherin inducer” includes any and all molecules capable of increasing specifically P-cadherin expression and/or P-cadherin function, such as binding β-catenin and/or other cellular skeleton components.

As used herein, the phrase “hair growth modulator” includes any and all molecules capable of increasing (e.g., accelerating) or decreasing (e.g., suppressing) hair growth.

As used herein, the phrase “hair growth inhibitor” includes any and all molecules capable of decreasing or suppressing hair growth.

As used herein, the phrase “hair growth inducer” includes any and all molecules capable of increasing or accelerating hair growth.

Several assays are known for monitoring P-cadherin function, such as binding β-catenin and/or other cellular skeleton components. These assays include immunoprecipitation of cell extracts with an anti-Pcadherin antibody and immunoblotting of this reaction products to reveal a 116 kD band representing P-cadherin as well as three smaller bands corresponding in decreasing size order to α-, β-, γ-catenins; microscopic examination of cell cultures in the presence of anti-E cadherin in which further inhibition of P-cadherin function leads to cell-cell interaction disruption and inhibition of keratinocyte differentiation; inhibition of actin cytoskeleton formation under changing Ca⁺⁺ concentrations in keratinocyte cell-cultures (Lewis, J. E., Jensen, P. J. & Wheelock, M. J J. Invest. Dermatol. 102, 870-877 (1994)). According to one embodiment of the present invention, the P-cadherin modulator is an antisense oligonucleotide capable of specifically binding to P-cadherin gene, pre-messenger RNA or messenger RNA under physiological conditions and hence serves as a P-cadherin inhibitor, reducing its level of expression.

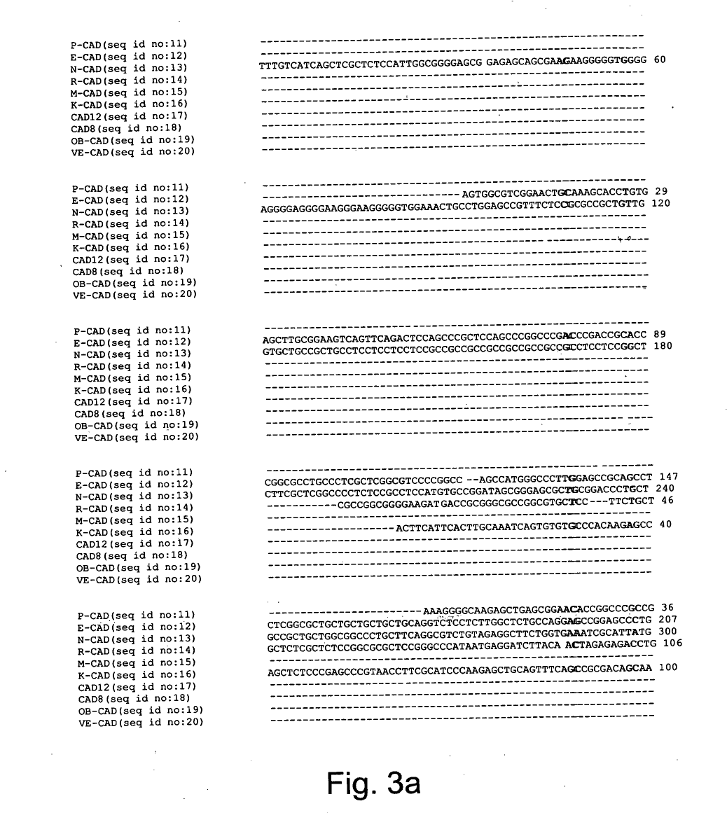

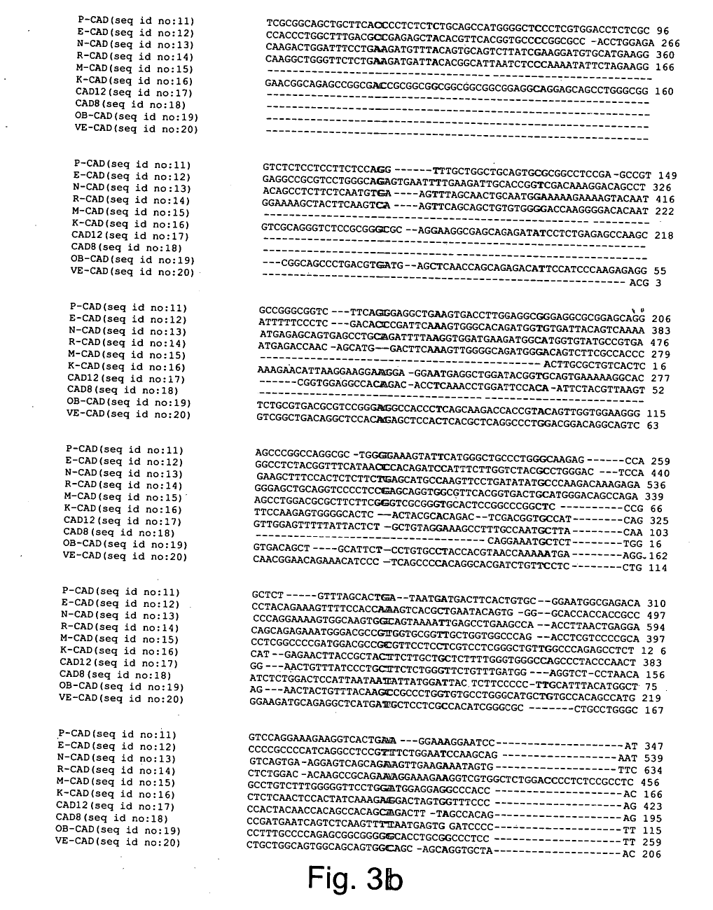

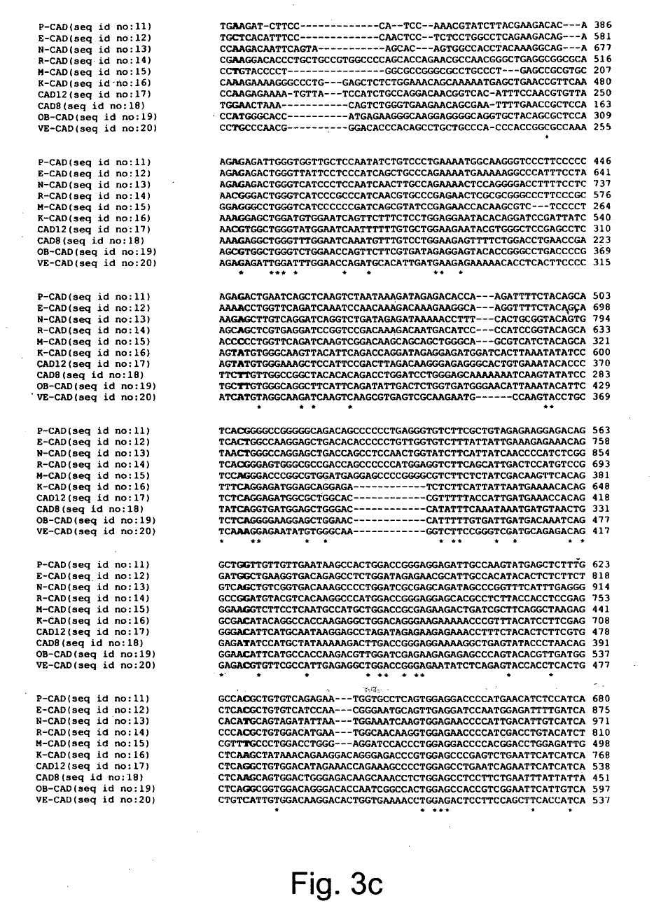

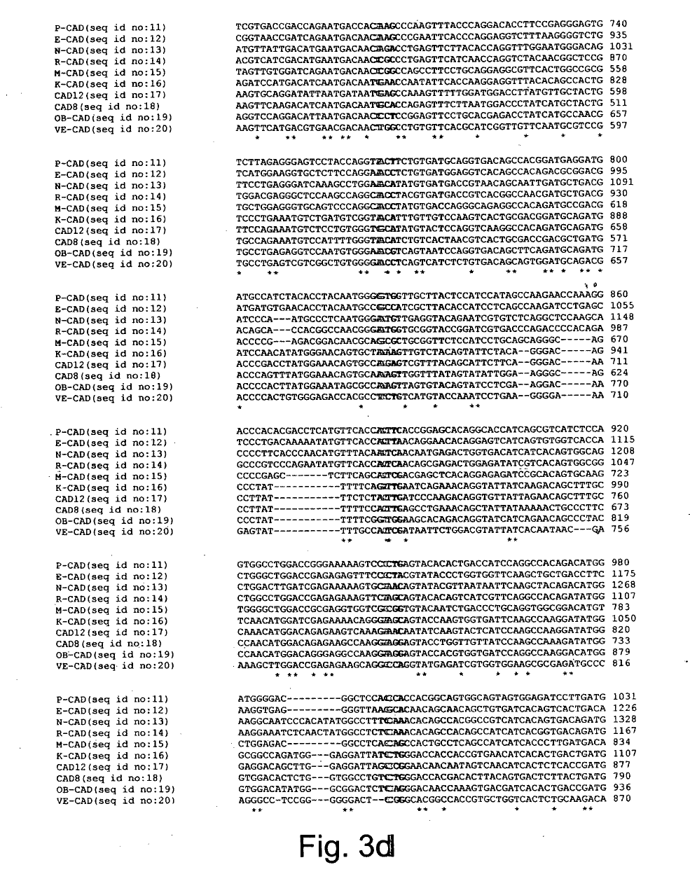

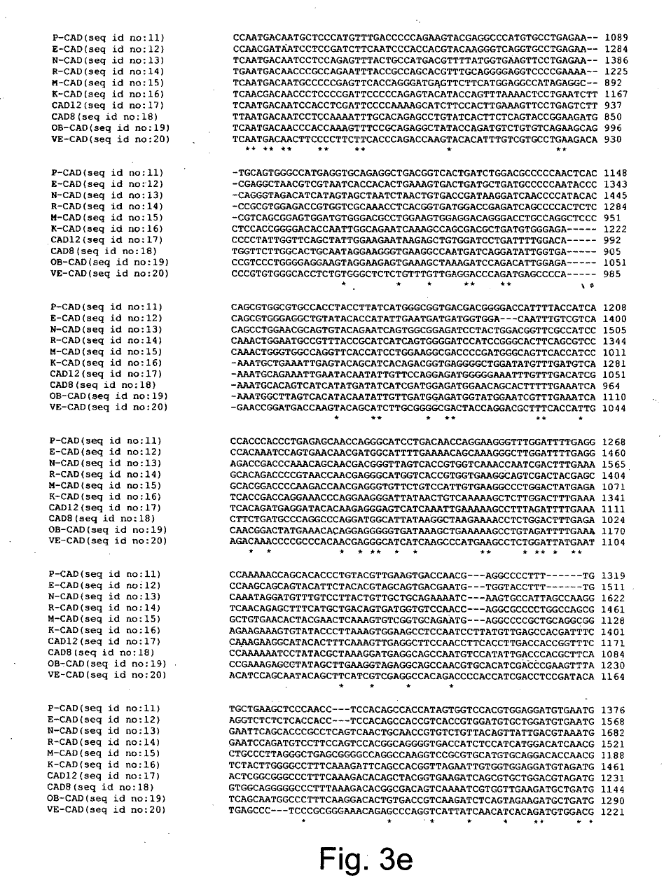

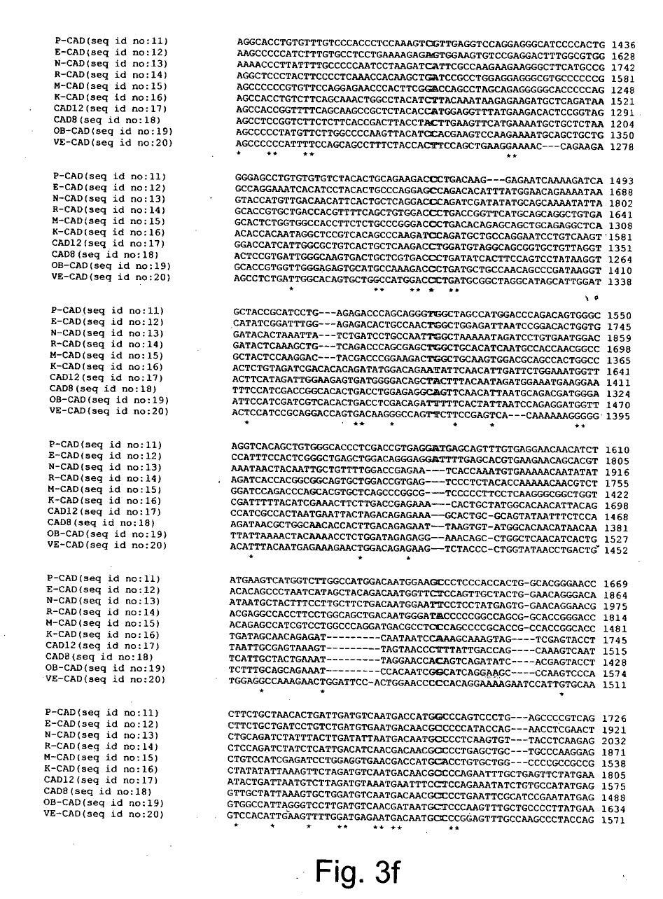

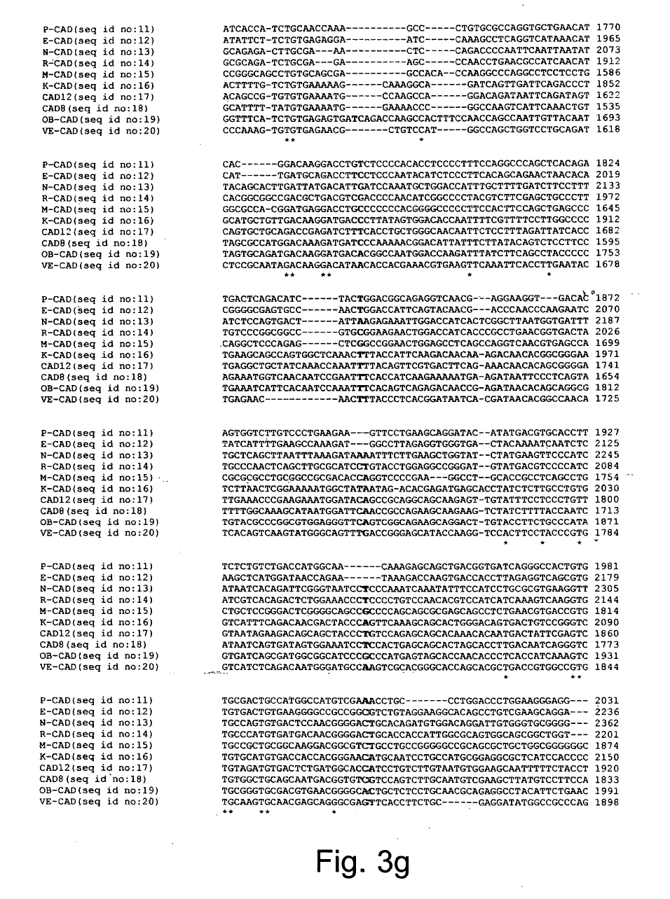

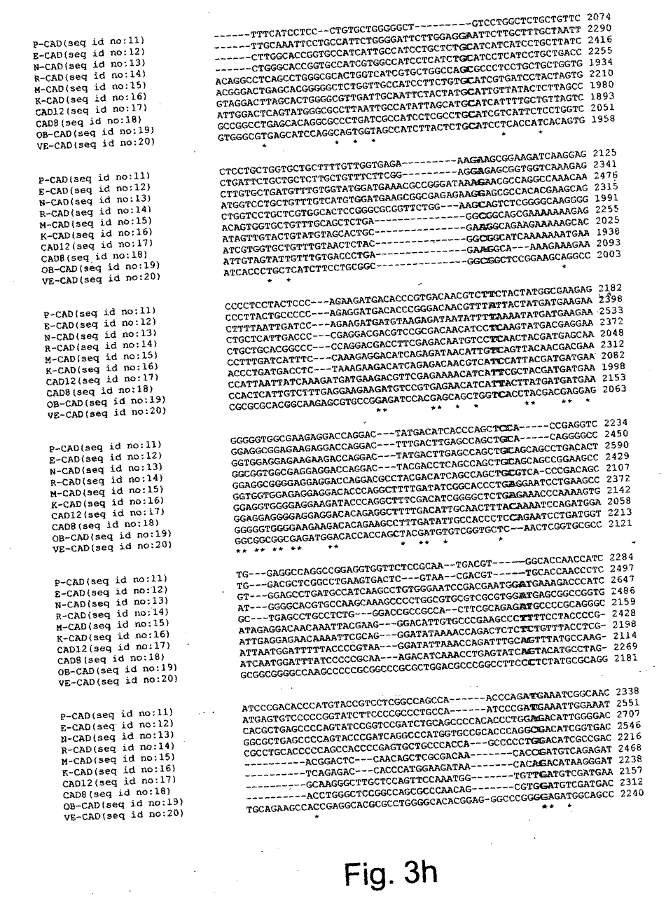

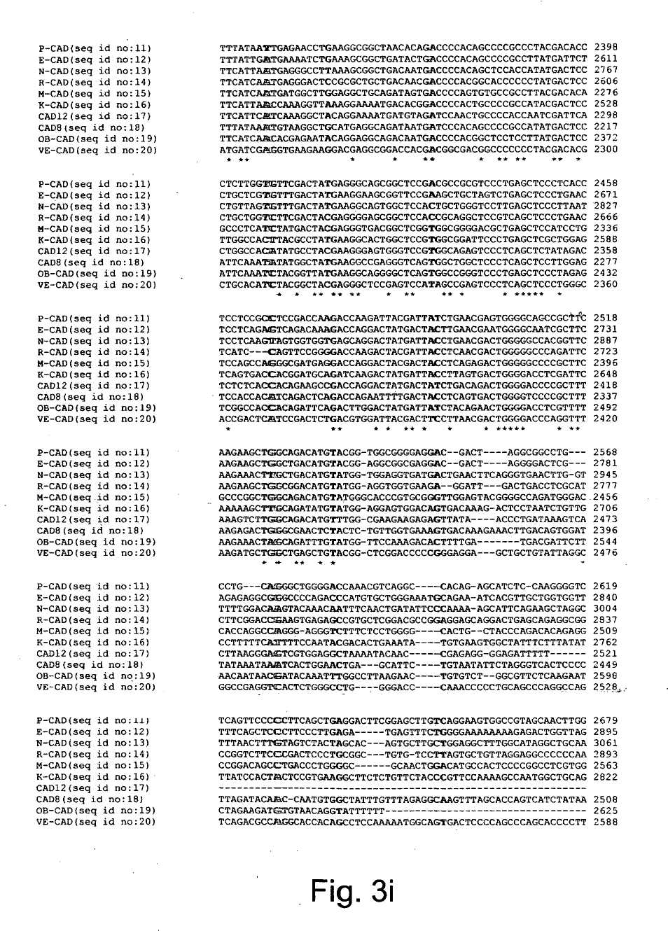

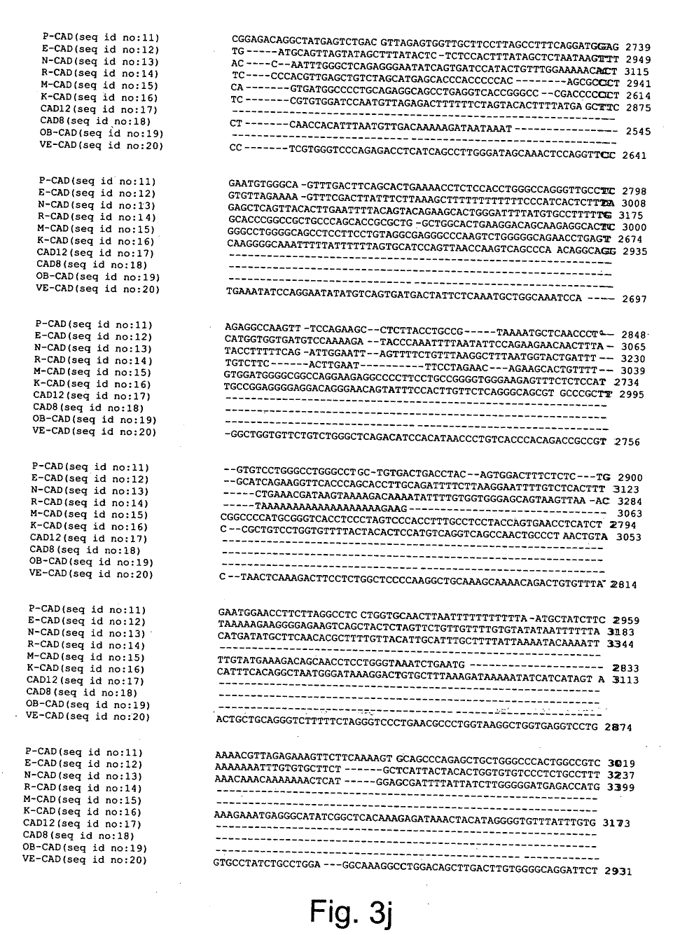

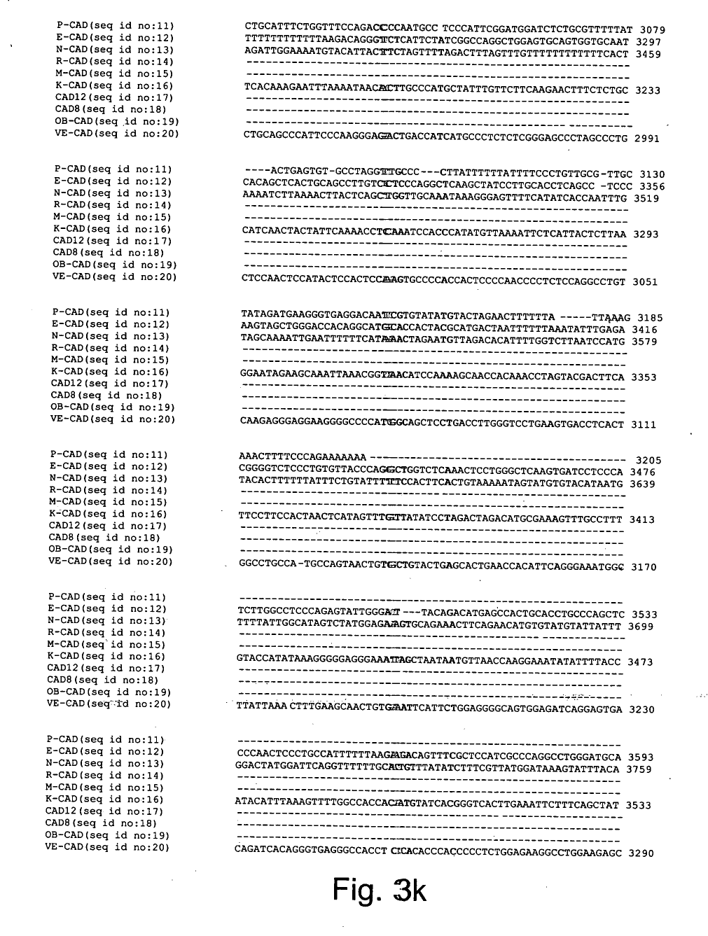

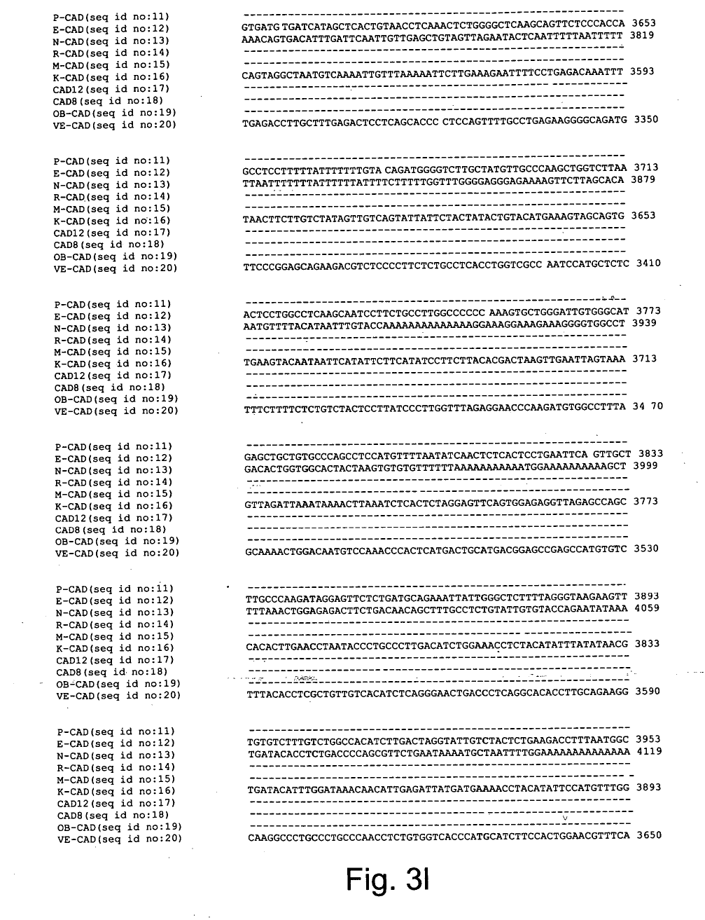

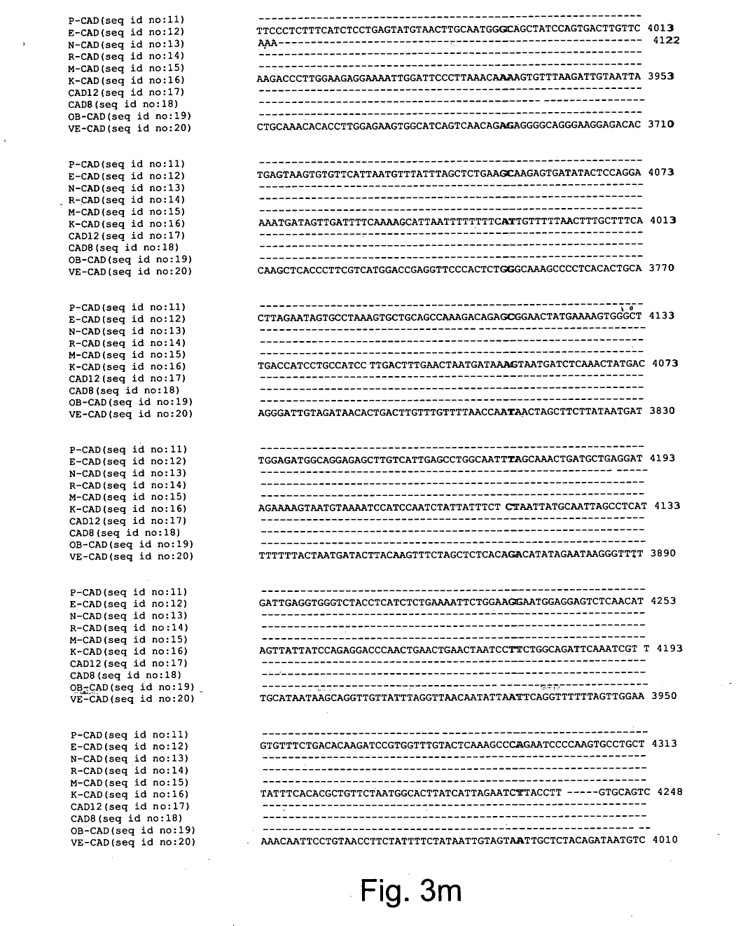

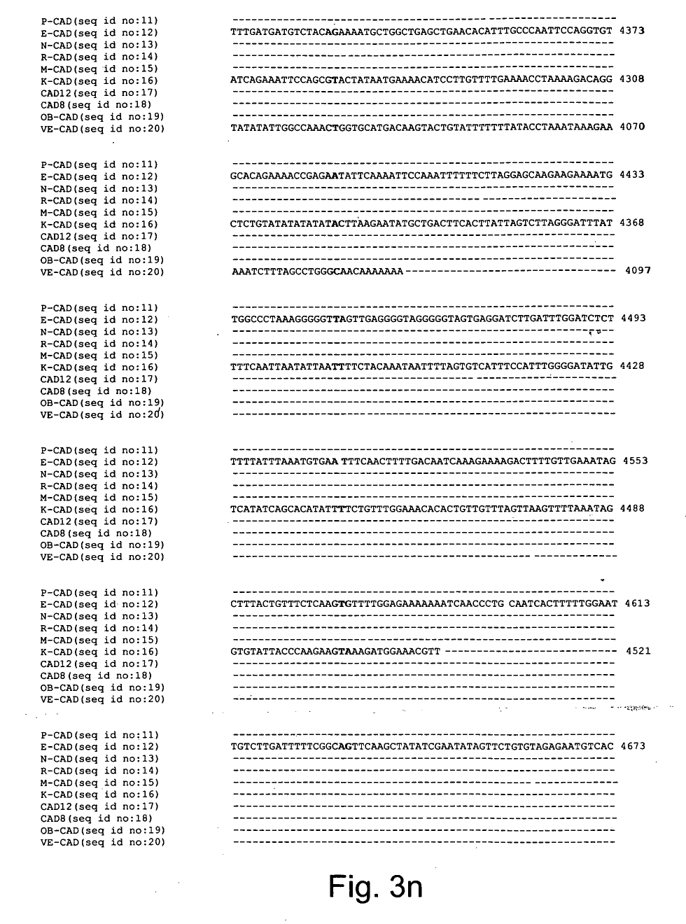

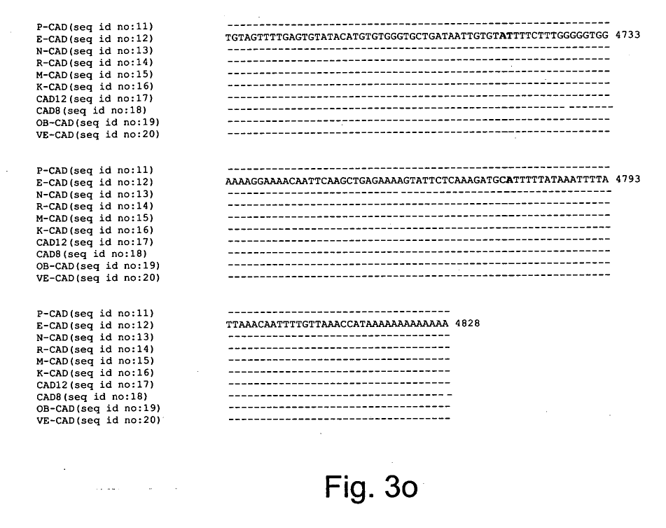

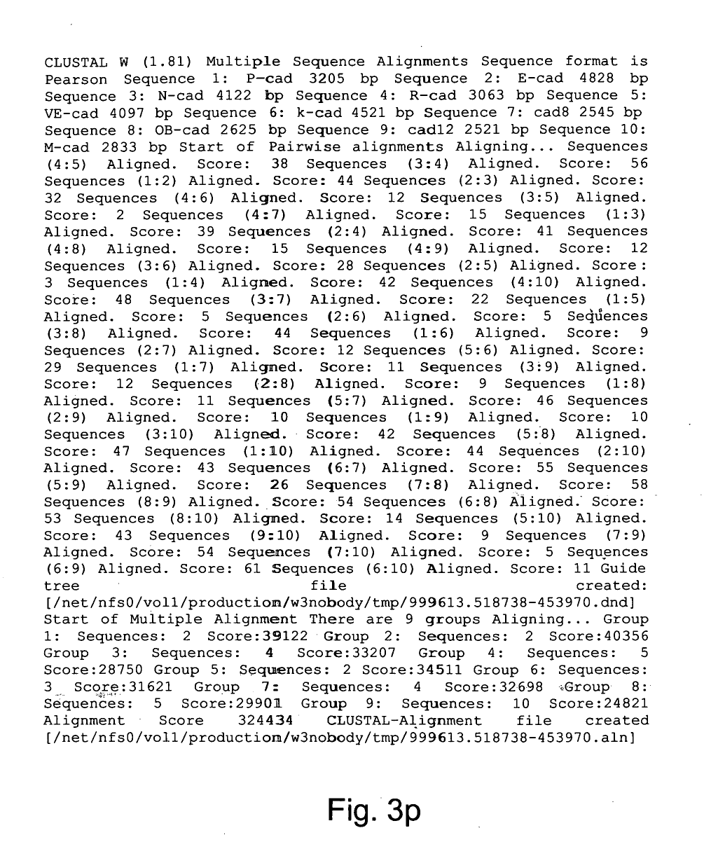

FIGS. 3a-p present an alignment of human cadherin cDNAs (SEQ ID NOs:11-20). Those regions for which no or low homology exists between P-cadherin and other human cadherins were identified. The following oligonucleotides are exemplary oligonucleotides capable of specifically binding to P-cadherin gene, pre-messenger RNA or messenger RNA under physiological conditions and hence serve as P-cadherin inhibitors, via inhibiting P-cadherin expression:

| | | | |
|---|---|---|---|
| 1. | GAGAGGTCCACGAGGGAGCCC | (74-94) | (SEQ ID NO:21) |
| 2. | CACGGCTCGGAGGCCGCGCA | (131-150) | (SEQ ID NO:22) |
| 3. | CGCCTCCAAGGTCACTTCAG | (171-191) | (SEQ ID NO:23) |
| 4. | CTAAACAGAGCTGGCTCTTG | (251-270) | (SEQ ID NO:24) |
| 5. | AGTGACCTTCTTTCCTGGAC | (311-330) | (SEQ ID NO:25) |
| 6. | GTTTGGATGGGAAGATCTTC | (349-368) | (SEQ ID NO:26) |
| 7. | CTTGTGTCTTCGTAAGATAC | (369-388) | (SEQ ID NO:27) |
| 8. | CTGGGGGAAGGGACCCTTGC | (429-448) | (SEQ ID NO:28) |
| 9. | CTTCAGCACAAAAGGGGCCT | (1308-1027) | (SEQ ID NO:29) |
| 10. | CAACGACTTTGGAGGGTGGGAC | (1391-1412) | (SEQ ID NO:30) |
| 11. | GTTGTTCCTCACAAACTGCTC | (1586-1606) | (SEQ ID NO:31) |
| 12. | GTGGTGGGAGGGCTTCCATTG | (1636-1656) | (SEQ ID NO:32) |
| 13 | GATCTGACGGGGCTCAGGGAC | (1709-1729) | (SEQ ID NO:33) |
| 14. | CATCTGTGAGCTGGGCCTGG | (1807-1826) | (SEQ ID NO:34) |
| 15. | CCTTCCTCGTTGACCTCTGCC | (1846-1866) | (SEQ ID NO:35) |
| 16. | CTTTGTTGCCATGGTCAGACAG | (1931-1952) | (SEQ ID NO:36) |
| 17. | GCAGCACCAGCAGGAGGAAC | (2071-2090) | (SEQ ID NO:37) |
| 18. | GGTTGGTGCCACGTCATTGCG | (2261-2281) | (SEQ ID NO:38) |
| 19. | GTTGGCTGGCCGAGGACGGTAC | (2278-2298) | (SEQ ID NO:39) |

As used herein, unless otherwise indicated, the term “antisense” or “antisense therapeutic” refers to oligonucleotides, modified oligonucleotides or other chemical compositions that bind in a sequence specific manner to a specified gene, its pre-mRNA, or its mRNA.

As used herein, unless otherwise indicated, the term “oligonucleotide” includes both oligomers of ribonucleotides, i.e., oligoribonucleotides, and oligomers of deoxyribonucleotides, i.e., oligodeoxyribonucleotides or oligodeoxynucleotides.

Unless otherwise indicated, the term “oligonucleotide” also includes oligomers that may be large enough to be termed “polynucleotides.”

The terms “oligonucleotide”, “oligodeoxynucleotide” and “oligodeoxyribonucleotide” include oligomers and polymers of the biologically significant nucleotides, adenine, deoxyadenine, guanine, deoxyguanine, thymidine, uridine, cytosine and deoxycytosine, as well as oligomers and polymers that contain other novel nucleotides and are capable of forming hybrids with the mRNA transcripts that encode P-cadherin. These terms also include oligomers and polymers having one or more purine or pyrimidine moieties, sugar moieties, or internucleotide linkage(s) that have been chemically modified. These terms include any oligomers and polymers that are composed of nucleotides or nucleotides containing any modifications listed above which also contain bases or modified bases that are joined to sugar moieties in the alpha and not the beta configuration (known in the art as “alpha anomers”) or any oligonucleotide or polynucleotide that contains one or more of these modifications. The oligonucleotides can be linear or circular and include oligomers that are modified at the 5′-end, 3′-end, or anywhere in the middle of the chain. Modifications may also involve the backbone or may occur through the nucleobases with reporter groups. These reporter groups can be lipids, phospholipids, sugarlipids, etherlipids, peptides, ligands to known or unknown receptors or any other hydrophobic moiety that can enhance or regulate the cellular uptake or the targeting of the oligonucleotide to a particular cell type. The reporter groups can also be a cross-linking group that can form covalent linkages between the oligonucleotide and the targeted mRNA with or without biological or chemical activation. The sugar-phosphate backbone can be joined by 3′-5′ or 2′-5′ linkages. The backbone modifications of the oligonucleotides may include those known in the art including phosphotriesters, methylphosphonates, phosphodiesters or phosphorothioates and also such backbone modifications which are based on peptides or any other non-phosphate linkages that are currently being employed or might be used by those skilled in the art. These terms also include any oligomer or polymer that has nucleosides, whether natural or containing modifications, that are joined together in linkages that are not 3′-5′, such as 3′-2′ phosphodiester, 5′-2′ phosphodiester, or phosphorothioate linkages.

The term “downstream” is used herein to indicate the 5′-3′ direction in a nucleotide sequence. Similarly, the term “upstream” indicates the 3′-5′ direction.

Unless otherwise indicated, the term “mRNA” is used herein to indicate either the mature or processed messenger RNA, or the unprocessed nuclear pre-mRNA that encodes the human P-cadherin.

Antisense oligodeoxynucleotides or ribozymes have been successfully employed to decrease mRNA translation (van der Krol, et. al., 1988; Cohen, 1991; Calabretta, 1991; Calabretta, et. al., 1991; Saison-Behmoraras, et. al., 1991). Once the oligonucleotides are taken up by the cells they can elicit an antisense effect by binding to the correct sequences on the target mRNA. The concept behind antisense therapy is based on the assumption that antisense oligonucleotides are taken up by cells and interact with a specific mRNA resulting in the formation of a stable heteroduplex. The interaction of the antisense oligonucleotide with its target mRNA is highly specific and is determined by the sequence of bases complementary to the antisense oligonucleotide as determined by Watson/Crick base pairing.

Antisense oligonucleotides used for therapeutic purposes were first proposed in 1978 by M. L Stephenson and P. C. Zamecnik (PNAS 75: 280-284). The concept behind antisense therapy relies on the ability of antisense oligonucleotides to be taken up by cells and form a stable heteroduplex with the target mRNA, thereby down regulating the targeted protein's synthesis.

It has been demonstrated in a number of systems by a number of investigators that oligonucleotides containing an antisense sequence targeting a portion of a particular mRNA are capable of hybridizing to the mRNA and inhibiting the translation of the transcript.

The interaction of an antisense oligonucleotide with target mRNA is highly specific, as hybridization is determined by the sequence of bases complementary to the antisense oligonucleotide (Watson/Crick base pairing of the two strands of nucleic acid). This results in multiple points of contact between the antisense oligonucleotide and the mRNA target, which increases the specificity for hybridization to the correct sequence.

Evidence for down regulation of protein synthesis by antisense oligonucleotides has been well documented in vitro (for reviews see van der Krol, A. R., et al. BioTechniques 6: 958-976, 1988; Milligen et. al. J. Med. Chem 36:1923-1937, 1993). In vivo studies using antisense oligonucleotides have demonstrated that injection of radiolabeled antisense oligonucleotides into the blood of mice results in distribution of full-length labeled oligonucleotide to the various tissues. Once in the tissue, oligonucleotides can elicit an antisense effect by binding to the correct mRNA and, thus, be suitable for a therapeutic (Miller, P. S. and Ts'o, P. O. P. Anticancer Drug Design 2: 117-128, 1987).

An example of antisense alopecia therapy is known in the art. The development and progression of androgenic alopecia is associated with the local accumulation of DHT. The enzyme steroid 5α-reductase type 1 is expressed in the inner epithelial sheath of the hair follicle. This enzyme functions to catalyze the conversion of testosterone to dihydrotestosterone. U.S. Pat. No. 5,994,319 teaches that antisense inhibition of steroid 5α-reductase type 1 expression, alone or in combination with other agents that decrease steroid 5α-reductase activity (i.e. Propecia™) or through the inhibition of the expression of other steroid 5α-reductase genes, is an effective means for treating androgenic alopecia.

Antisense therapy, is used according to the present invention, alone or in combination of other hair growth inhibitors or hair removers to inhibit hair growth by selectively binding to P-cadherin nucleic acids (e.g., pre-mRNA, m-RNA or gene encoding P-cadherin), thereby inhibiting P-cadherin expression and inhibiting hair growth.

Antisense oligonucleotides (at a concentration of 0.01 μg to 100 g per kg/body weight) capable of down regulating the expression of P-cadherin is administered to patients at locations where hair removal is desired in a topical application optionally containing at least one additional hair growth inhibitor or hair remover substance.

Recent evidence suggests that it is possible to deliver DNA molecules to the hair follicle by using the hair shaft appendage as an integral component of the delivery strategy (Li L, Hoffman RM. (1995) The feasibility of targeted selective gene therapy of the hair follicle. Nat Med. 1995 July; 1(7):705-6). The formulation used for delivery can be comprised of any suitable delivery vehicle that is compatible with the physical properties of antisense oligonucleotides. For example, such agents are soluble in a solution of 60% ethanol, propylene glycol, water and, thus, the formulation may be comprised of these components. Additionally, various liposomal formulations may be added to the delivery vehicle to promote delivery to the hair follicle.

The oligonucleotides of the present invention can be constructed and purified by methods known in the art. The specific oligonucleotide sequences are constructed so as to have a nucleotide sequence that is complementary to a nucleotide sequence that comprises a portion of the gene that encode human P-cadherin. The described sequences are most often 21 bases in length but may include as few as 3 bases, typically, at least 10, 11, 12, 13, 14, 15, 16, 17, 18, 19, 20, 21, 22, 23, 24 or at least 25-40 bases and as many as 100 bases or more. The targeted sequences have been selected because it is believed that they are essential for the translation of the P-cadherin transcript. The oligonucleotides of the present invention have been selected because they are capable of hybridizing with a high degree of specificity to regions of the transcript including the translation initiation site along with sequences 5′ or 3′ to the translation initiation site. Other oligonucleotides may be selected that hybridize to the 5′ cap region of the mRNA or sequences 3′ or 5′ to the cap site. Additional oligonucleotide sequences of the present invention are complementary to sequences found in the 3′ untranslated region of the P-cadherin gene and are unique to the P-cadherin gene. Such sequences are capable of hybridizing with specificity to sequences found in the 3′-untranslated region of the P-cadherin mRNA transcripts. In addition to the sequences described above, other sequences contained within the P-cadherin transcript are targeted. This strategy has been adopted because, as yet, there is no method currently available that can predict, with precision, sequences that will become effective therapeutics. Moreover, this invention further contemplates antisense oligonucleotides made complementary to any portion of the P-cadherin gene and which are capable of cross-linking DNA, intercalating DNA or binding more tightly by mechanisms such as, for example, triple stranding. Furthermore, the invention contemplates that any oligonucleotide capable of substantially inhibiting the expression of P-cadherin can be used.

Oligonucleotides of varying lengths have been successfully used to inhibit gene expression. For example, in U.S. Pat. No. 4,806,463 oligonucleotides ranging in size from 12 bases to 26 bases were shown to be incorporated by cells and to be capable of inhibiting the expression of a target mRNA.

In order for the described antisense oligonucleotides to function therapeutically, the oligonucleotides or modified oligonucleotides must be taken up by the cell that expresses the target gene, pre-mRNA, or mRNA. The oligonucleotides of the present invention are constructed so as to ensure that the oligonucleotide will pass through the plasma membrane and achieve an intracellular concentration that is sufficient to decrease the expression of P-cadherin.

Oligonucleotides that are constructed to bind to the P-cadherin gene are further modified, if necessary, to enable them to pass through the nuclear membrane in levels that are sufficient to reduce transcription. Recent attempts at enhancing the cellular uptake of antisense oligonucleotides have employed a wide variety of techniques including the use of lipoproteins, and a wide variety of conjugates, such as poly-L-lysine, polyethylene glycol and cholesterol.

Conjugation of cholesterol to the 5′ end of an oligonucleotide has been reported to result in a molecule that exhibited reduced serum clearance due to reduction in renal excretion, compared to that observed with control oligodeoxynucleotides. As a result, the conjugation of cholesterol to deoxynucleotides may allow an increase in the delivery of drug to liver cells via the LDL transport mechanism. Liposomes containing antisense oligonucleotides can also be targeted to specific cell types by the addition of cell-specific antibodies. These and other methods of achieving and maintaining adequate intracellular concentrations of the oligonucleotides are contemplated by this invention and include other methods and compositions that have the capacity to enhance cellular uptake or decrease the efflux of internalized oligonucleotides. Such modifications should not alter the specificity of the oligonucleotide for its target sequence.

Antisense oligonucleotides that are intended for use as drugs must achieve sufficient concentrations in order to decrease the expression of a target protein in a manner that provides therapeutic benefit. The oligonucleotides contemplated in this invention are constructed, or otherwise modified, so as to increase their stability by enhancing resistance to various degradative enzymes (e.g., nucleases). Such modifications will function to permit the concentration of the oligonucleotide therapeutic to be maintained at a level that is sufficient so as to realize therapeutic benefit but cannot substantially alter the specificity of the oligonucleotide for its target sequence. Modifications that improve oligonucleotide stability or efficacy include but are not limited to modifications to the phosphate backbone, termini, sugar moieties and the individual nucleic acid bases. Conjugations to peptides, proteins, carbohydrates, lipids, vitamins or any other conjugation that increases therapeutic potency or efficacy can also be used. Also, any modifications resulting in stable secondary structures including circularization of the oligonucleotide and target sequence, and intrastrand joining of the 3′ to the 5′ termini through covalent bonds or hybridization and triple stranded binding to mRNA can also be made. Any modifications that reduce nuclease sensitivity while substantially maintaining the affinity and substrate specifically and solubility exhibited by unmodified oligonucleotides are within the scope of the invention.

Several chemically modified oligonucleotides have been developed which substantially block or improve resistance to nuclease activity. These oligonucleotide modifications include phosphorothioate oligonucleotides wherein one of the phosphate oxygens is replaced by sulfur. Another type of modification of oligonucleotides is accomplished by replacing the charged phosphate oxygen with a methyl group or other alkyl group. These nonionic DNA analogs include, for example, methyl phosphonates, alkyl-phosphorothioates, and O-alkyl phosphotriesters. A preferred O-alkyl phosphotriester is O-methylphosphotriester. Other DNA backbone modifications at the phosphate group include for example, phosphorodithioate, and phosphotriester oligonucleotides or oligonucleotides based on protein-nucleic acid structures or morpholino-like structures.

Various chemical modifications to either or both the 3′- or 5′-termini and the individual nucleic acid bases are known to improve stability of oligonucleotides to nucleases, stabilize the interaction of oligonucleotides with their specific target molecule, or enhance uptake of the oligonucleotides by cells. Moreover, chemical modifications to the 3′ or 5′ termini or modifications internal to the oligonucleotide can also be introduced as reporter molecules for example, to allow tracking of the oligonucleotide or as lipophilic moieties to enhance cell uptake. Such molecules can be introduced to both unmodified and backbone modified synthetic oligonucleotides. These moieties can be introduced for example, through thio or amino linkages to terminal hydroxyl or phosphate groups or to specific bases.

Other modifications to the oligonucleotides contemplated in this invention include for example, DNA intercalators, photochemically activated cross-linking or cleaving agents, alkylating agents and redox active nucleic acid cleaving groups.

In vivo and in vitro studies of the degradation of chemically modified oligonucleotides have clearly illustrated that modifications to the phosphate backbone, termini, sugar moiety and individual nucleic acids improve oligonucleotide efficacy or stability or both. Moreover, acute toxicity studies in mice have demonstrated that some modified oligomers are tolerated at about the same concentrations without undesirable side effects as unmodified oligomers.

Regardless of the modifications that are contemplated by this invention, a successful antisense therapeutic that is designed to inhibit the expression of P-cadherin must hybridize with sufficient specificity so as to reduce the potential of non-mechanistic-based toxicity. Investigations into the toxicity of other antisense oligonucleotides have not revealed significant damage or lethality to cells. To date, in vitro studies examining toxicity of antisense oligonucleotides have been limited primarily to modified oligomers wherein the phosphodiester linkages between the nucleosides have been replaced with either phosphorothioates or methylphosphonates. Under the conditions tested, exposure of a variety of cell lines to phosphorothioate oligomers has not resulted in any significant toxicity.

Antisense oligonucleotides are one way of delivering antisense therapy. However, antisense gene therapy, whereby a nucleic acid construct encoding an antisense transcript is used to introduce antisense therapy into cells. Hence, according to another embodiment of the present invention the P-cadherin inhibitor is an antisense construct encoding an antisense transcript capable of specifically binding to P-cadherin gene, pre-messenger RNA or messenger RNA under physiological conditions.

On the other hand, gene therapy can also be used in accordance with the teachings of the present invention to express or overexpress P-cadherin in hair follicle cells of alopecia patients in order to induce hair growth. Hence, according to another embodiment of the present invention the P-cadherin modulator is a polynucleotide capable of directing P-cadherin expression in hair follicle cells and hence serves as a P-cadherin inducer.

Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional (sense) RNA, antisense RNA, ribozyme, etc.) whose production in vivo is desired. For example, the genetic material of interest can encode a P-cadherin protein, a peptide capable of binding P-cadherin and modulate its function, a functional (sense) P-cadherin RNA, antisense P-cadherin RNA, P-cadherin ribozyme, etc. For review see, in general, the text “Gene Therapy” (Advanced in Pharmacology 40, Academic Press, 1997).

In vivo gene therapy (as opposed to ex vivo gene therapy), the genetic material to be transferred into the cells is introduced into the cells of the recipient organism in situ, that is within the recipient. In an alternative embodiment, if the host gene is defective, the gene is repaired in situ (Culver, 1998. (Abstract) Antisense DNA & RNA based therapeutics, February 1998, Coronado, Calif.). These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any nontranslated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York 1989, 1992), in Ausubel et al., Current Protocols in Molecular Biology, John Wiley and Sons, Baltimore, Md. 1989), Chang et al., Somatic Gene Therapy, CRC Press, Ann Arbor, Mich. 1995), Vega et al., Gene Targeting, CRC Press, Ann Arbor Mich. (995), Vectors: A Survey of Molecular Cloning Vectors and Their Uses, Butterworths, Boston Mass. 1988) and Gilboa et al. (Biotechniques 4 (6): 504-512, 1986) and include, for example, stable or transient transfection, lipofection, electroporation and infection with recombinant viral vectors.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo. Viral vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor which includes cells of epithelial origin as well as others. This vector as well as others that exhibit similar desired functions can be used to treat a mixed population of cells and can include, for example, a tissue, e.g., skin tissue, or a human subject.

Features that limit expression to particular cell types can also be included. Such features include, for example, promoter and regulatory elements that are specific for the desired cell type. The P-cadherin promoter can be used to direct gene expression in hair follicle cells.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in may cases, to elude host defense mechanisms. Typically, viruses infect and propagate in specific cell types. The targeting specificity of viral utilizes its natural specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject.

According to another embodiment of the present invention, the P-cadherin modulator, or the molecule capable of binding P-cadherin, is an anti-P-cadherin antibody and hence serves as a P-cadherin inhibitor.

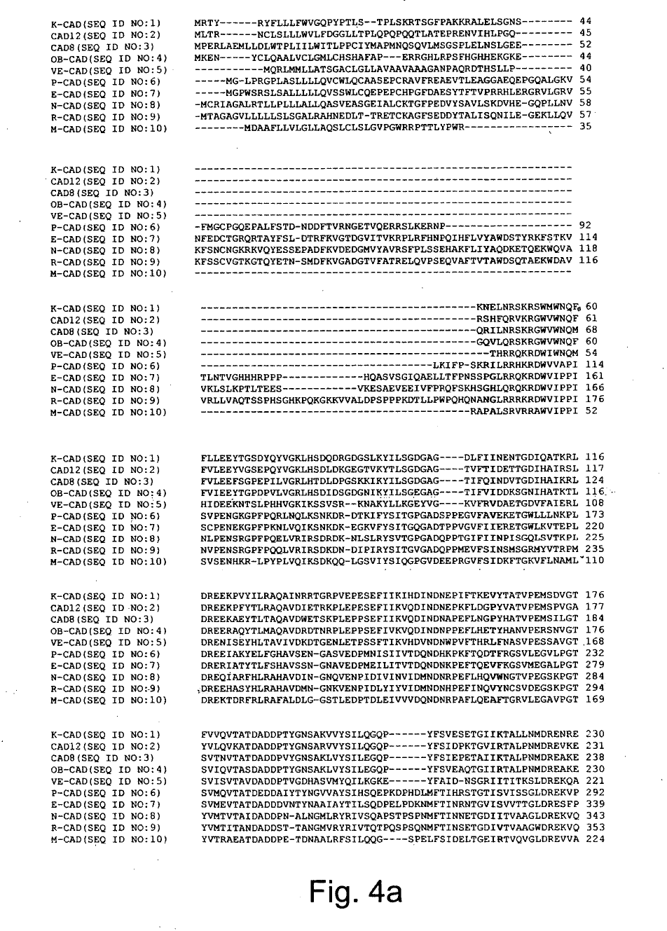

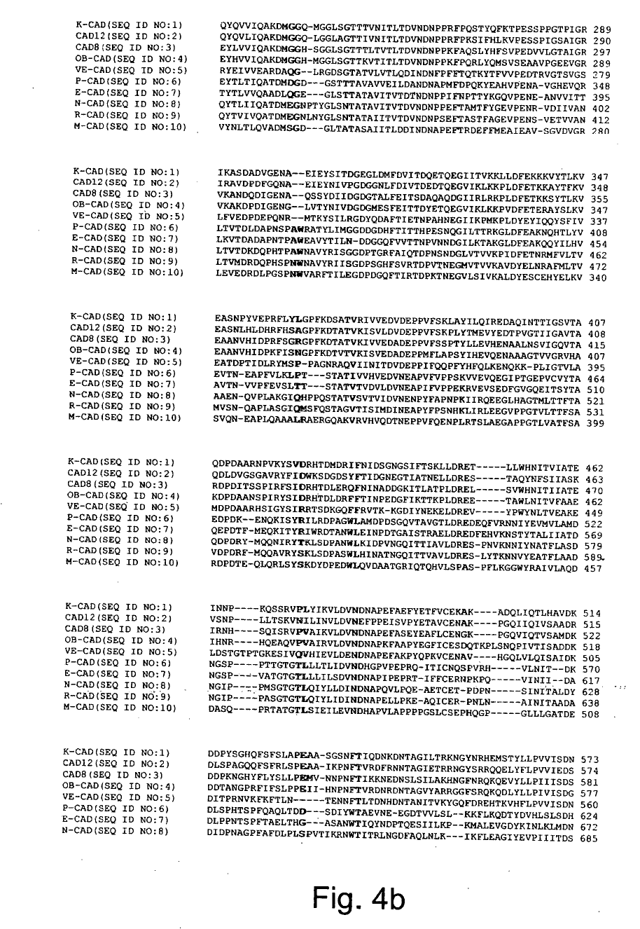

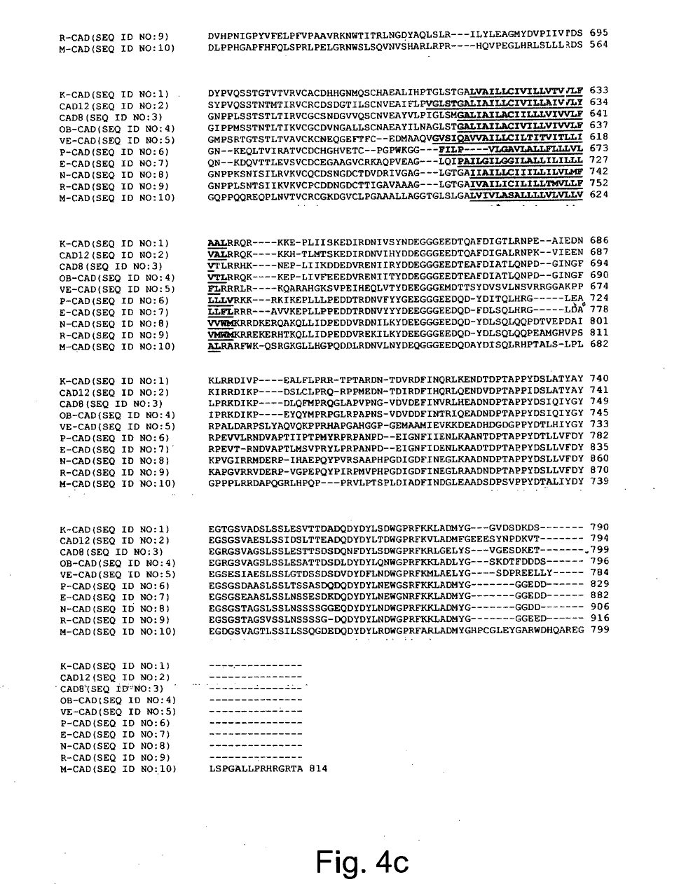

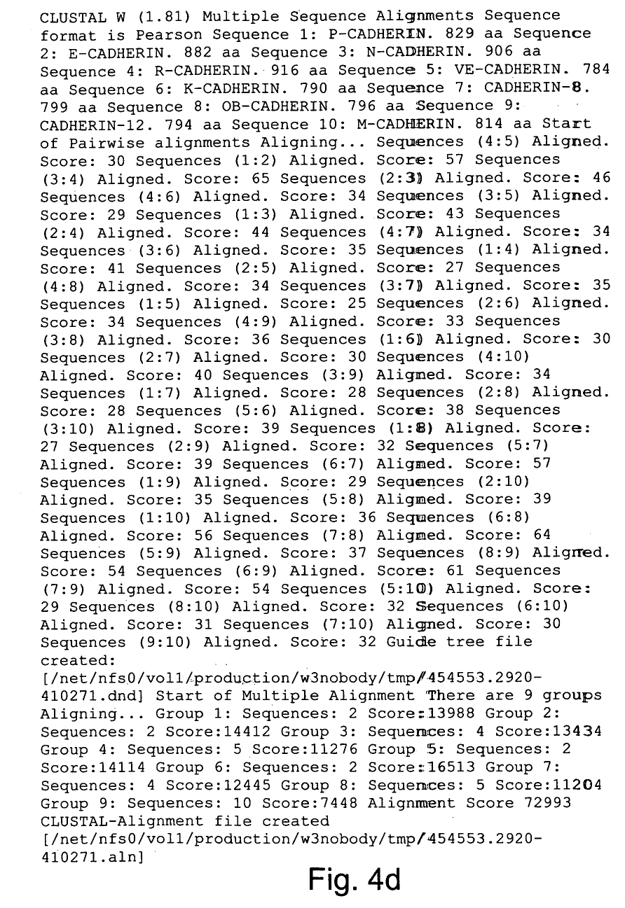

FIGS. 4a-d shows an alignment of the intracellular and extracellular portions of human cadherins. Short sequences of low similarity between P-cadherin and the other human cadherins, especially E-cadherin, were identified. These sequences are used in accordance with the teachings of the present invention to generate antibodies specific to P-cadherin.

The following peptides are thought to have a potential of eliciting antibodies specific to P-cadherin as they share low or no similarity with corresponding sequences of other human cadherins and/or mouse cadherins and were identified as immunogenic by the peptidestructure algorithm from the GCG package:

For the extracellular domain of P-cadherin:

1. VPENGKGPFP (117-124) (SEQ ID NO:40) both immunogenic and not homologous to either mouse P-cadherin or other human cadherins;

| | | |
|---|---|---|
| 2. QEPKDPHDLMFTIHRSTGT | (259-277); | (SEQ ID NO:41) |
| 3. DNGSPPTTGT | (522-531); | (SEQ ID NO:42) |
| 4. TDKDLSPHTSPFQAQLTDDSDIY | (568-590); | (SEQ ID NO:43) |
| 5. DCHGHVETCPGPWKGG | (639-654); | (SEQ ID NO:44) |

For the cytoplasmic domain of P-cadherin:

| | | |
|---|---|---|
| 6. MYRPRPANPDEI | (743-754) | (SEQ ID NO:45) |

These or similar peptides are used according to the present invention to elicit P-cadherin specific antibodies which are used for inhibiting hair growth by topical application onto the skin in a formulation that enhances the penetration of such antibodies into cells of the hair follicle.

As used herein, the term “antibody” includes any monoclonal or polyclonal immunoglobulin, or a fragment of an immunoglobin such as sFv (single chain antigen binding protein), Fab1 or Fab2. The immunoglobulin could also be a “humanized”, in which murine variable regions are fused to human constant regions, or in which murine complementarity-determining regions are grafted onto a human antibody structure (Wilder, R. B. et al., J. Clin. Oncol., 14:1383-1400, 1996). Unlike mouse or rabbit antibodies, “humanized” antibodies often do not undergo an undesirable reaction with the immune system of the subject. The terms “sFv” and “single chain antigen binding protein” refer to a type of a fragment of an immunoglobulin, an example of which is sFv CC49 (Larson, S. M. et al., Cancer, 80:2458-68, 1997).

The elicitation of an anti-P-cadherin antibody is through in vivo or in vitro techniques, the antibody having been prepared by a process comprising the steps of (a) exposing cells capable of producing antibodies to P-cadherin or an immunological part thereof (e.g., a peptide fragment or synthetic peptide derived therefrom) and thereby generating antibody producing cells; (b) immortalizing the antibody producing cells by, for example, either fusing the antibody producing cells with myeloma cells or infecting the antibody producing cells with an immortalizing (transforming) virus and thereby generating a plurality of immortalized (e.g., transformed or hybridoma) cells each producing a monoclonal antibody; and (c) screening a plurality of monoclonal antibodies to identify a monoclonal antibody which specifically binds P-cadherin.

The cDNA encoding the monoclonal antibody can then be isolated by conventional techniques (e.g., screening a cDNA library with a probe that hybridizes to the portion encoding the constant region of the antibody). Portions of the cDNA encoding the variable regions of the antibody can be fused in-frame to other polypeptides such as the constant region of an antibody derived from a human being, to thereby obtain a humanized single chain antibody.

In another approach a phage display library presenting variable regions of antibodies fused to one or more of their coat proteins is enriched for those phages presenting antibodies that bind P-cadherin. Individual phage clones are then isolated and their genetic material sequenced to determine the amino acid sequence of the antibody they display. Then, a corresponding peptide is synthesized using solid phase techniques and tested for binding P-cadherin. General protocols for antibody-phage display technology are available from the Pharmacia Biotech (Uppsala, Sweden) Recombinant Phage Antibody System (RPAS).

Methods of generating, screening and characterizing the specificity of binding of an antibody are well known in the art. Further insight on these topics is available in, for example, “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219, 5,011,771 and 5,281,521.

Antibodies that are constructed to bind to P-cadherin may be further modified, if necessary, to enable them to pass through the cell membrane in levels that are sufficient to reduce P-cadherin function. Recent attempts at enhancing the cellular uptake of antibodies have employed a wide variety of techniques including the use of lipoproteins, polyethylene glycol and cholesterol. Liposomes containing antibodies can also be targeted to specific cell types by the addition of cell-specific antibodies on the outside of the liposome structure. These and other methods of achieving and maintaining adequate intracellular concentrations of the antibodies are contemplated by this invention and include other methods and compositions that have the capacity to enhance cellular uptake or decrease the efflux of internalized antibodies. Such modifications should not alter the specificity of the antibody for its target protein.

The present invention further contemplates the use of low molecular weight (e.g., up to 1,500 Da) organic compounds as either P-cadherin inhibitors or inducers as hair growth inducers or inhibitors, respectively. Chemical libraries of hundred of thousands of low molecular weight organic compounds are presently available on the market for use in highthroughput binding/screening assays. Such libraries can be screened for ligands that bind P-cadherin and modulate P-cadherin function. Such ligands can thereafter be tested in vivo to determine their effect on hair growth. Following the identification of a ligand as binding to P-cadherin, tests are conducted to establish whether it also modulates P-cadherin function (e.g., binding to β-catenin or other cellular skeleton components) and thereafter tests are conducted to establish whether it also modulates hair growth. Structure optimization and retesting are thereafter practiced to increase modulation activity. During structure optimization advantage can be taken of the 3D structure of P-cadherin. Similarly, rational drug design can take advantage of the 3D structure of P-cadherin.

Yet another type of candidate P-cadherin modulators are peptides. The present invention contemplates the use of a two hybrid system to identify peptides that specifically bind P-cadherin.

One approach for elucidating protein-protein binding in cells is the yeast-based two-hybrid system (Fields and Song (1989) Nature 340:245). That system utilizes chimeric genes and detects protein-protein interactions via the activation of reporter-gene expression. Reporter-gene expression occurs as a result of reconstitution of a functional transcription factor caused by the association of fusion proteins encoded by the chimeric genes. Typically, polynucleotides encoding two-hybrid proteins are constructed and introduced into a yeast host cell. The first hybrid protein consists of the yeast Gal4 DNA-binding domain fused to a polypeptide sequence of a known protein (often referred to as the “bait”). The second hybrid protein consists of the Gal4 activation domain fused to a polypeptide sequence of a second protein (often referred to as the “prey”). Binding between the two-hybrid proteins reconstitutes the Gal4 DNA-binding domain with the Gal4 activation domain, which leads to the transcriptional activation of a reporter gene (e.g., lacZ or HIS3), which is operably linked to a Gal4 binding site.

Homo- and heterodimeric protein complexes mediate many cellular processes and abnormal protein interactions underlie various medical conditions. Yan et al. (1995) Cancer-Res. 55: 3569-75. Research on such complexes has led to efforts to understand disease at the molecular level and to a search for small molecule effectors of such complexes. Such effectors could modulate protein interactions and are potential therapeutic agents. Gibbs & Oliff (1994) Cell 79: 193-198. Most often, such effectors have been identified using various biochemical and immunological in vitro approaches. The advantages of genetic approaches in drug discovery, however, have received increased attention. Liuzzi et al. (1994), Nature 372: 695-8. These advantages include both cost-effectiveness and simplicity. Several such genetic systems, in particular the yeast-two hybrid system, meets all these criteria and is also equally suitable for the detection of both homo- and heterodimeric protein interactions. Another unique feature of the yeast two-hybrid system is its ability to detect the desired protein-protein interaction without interference by competing interactions. Fields & Song (1989) Nature 340: 245-6. The system has been successfully used for the analysis of protein interactions and for the isolation of interacting proteins through interaction cloning. For a review, see Allen et al. (1995), Trends in Biochem. Sci. 20: 511-16.

Prokaryote two-hybrid systems are also available. E. coli strains can be hyperpermeable. Nakamura & Suganuma (1972) J. Bacteriol. 110: 329-35. One can use this hyperpermeability to maximize the number of small molecules that can be evaluated. In addition, E. coli has a rapid growth rate, permitting shorter turnaround times during drug screening. Furthermore, one can transform E. coli at high frequencies, facilitating interaction cloning. U.S. Pat. No. 6,051,381, teaches a prokaryote two-hybrid system. U.S. Pat. No. 6,251,676, teaches a mammalian two-hybrid system. Both of which are incorporated herein by reference.

In another approach a phage display library presenting short peptides (e.g., 6-8 amino acids) fused to one or more of the phage's coat proteins is enriched for those phages presenting peptides that bind P-cadherin. Individual phage clones are then isolated and their genetic material sequenced to determine the amino acid sequence of the short peptide they display. Then, a corresponding peptide is synthesized using solid phase techniques and tested for binding P-cadherin. Further insight regarding phage display libraries, their enrichment and screening is present in, for example, Frenkel and Solomon, J. of Neuroimmunol. 88:85-90,1998.

A peptide that binds P-cadherin can be an inhibitor or inducer of its activity. Once this is established, such a peptide is tested for hair growth modulation.

As used herein in the specification and in the claims section below the term “peptide” includes native peptides (either degradation products, synthetically synthesized peptides or recombinant peptides) and peptido-mimetics (typically, synthetically synthesized peptides), such as peptoids and semipeptoids which are peptide analogs, which may have, for example, modifications rendering the peptides more stable while in a body, or more immunogenic. Such modifications include, but are not limited to, cyclization, N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modification and residue modification. Methods for preparing peptido-mimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further detail in this respect are provided hereinunder.

Thus, a peptide according to the present invention can be a cyclic peptide. Cyclization can be obtained, for example, through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)ₙ—COOH)—C(R)H—COOH or H—N((CH₂)ₙ—COOH)—C(R)H—NH₂, wherein n=1-4, and further wherein R is any natural or non-natural side chain of an amino acid.

Cyclization via formation of S—S bonds through incorporation of two Cys residues is also possible. Additional side-chain to side chain cyclization can be obtained via formation of an interaction bond of the formula —(—CH₂—)ₙ—S—CH₂—C—, wherein n=1 or 2, which is possible, for example, through incorporation of Cys or homoCys and reaction of its free SH group with, e.g., bromoacetylated Lys, Orn, Dab or Dap.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as TIC, naphthylalanine (Nol), ring-methylated derivatives of Phe, halogenated derivatives of Phe or o-methyl-Tyr.

Tables 1-2 below list all the naturally occurring amino acids (Table 1) and non-conventional or modified amino acids (Table 2).

TABLE 1

| Amino Acid | Three-Letter Abbreviation | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid | Xaa | X |
| as above | | |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dmnser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dmnthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethlglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval | L-N-methylhomophenylalanine | Nmhphe |
| | Nnbhm | | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

A peptide according to the present invention can be used in a self standing form or be a part of a larger moiety such as a protein or a display moiety such as a display bacterium, a display phage or a display cell.

A peptide according to the present invention includes at least five, optionally at least six, optionally at least seven, optionally at least eight, optionally at least nine, optionally at least ten, optionally at least eleven, optionally at least twelve, optionally at least thirteen, optionally at least fourteen, optionally at least fifteen, optionally at least sixteen or optionally at least seventeen, optionally between seventeen and twenty five or optionally between twenty five and at least thirty amino acid residues (also referred to herein interchangeably as amino acids).

Accordingly, as used herein the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and omithine. Furthermore, the term “amino acid” includes both D- and L-amino acids.

According to an additional aspect of the present invention there is provided a hair growth modulator identified by the methods described herein.

According to yet an additional aspect of the present invention there is provided a method of modulating hair growth comprising administering to a subject in need a therapeutically effective amount of the hair growth modulator described herein.

A compound (active ingredient) according to the present invention can be administered to an organism, such as a human being or any other mammal, per se, or in a pharmaceutical composition where it is mixed with suitable carriers or excipients.

As used herein a “pharmaceutical composition” refers to a preparation of one or more of the compounds described herein, or physiologically acceptable salts or prodrugs thereof, with other chemical components such as physiologically suitable carriers and excipients. The purpose of a pharmaceutical composition is to facilitate administration of a compound to an organism. In particular, the purpose of a pharmaceutical composition in accordance with the present invention is to facilitate administration of a compound to the skin organism, specifically to hair follicles.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Pharmaceutical compositions may also include one or more additional active ingredients, such as, but not limited to, anti inflammatory agents, antimicrobial agents, vitamins, anesthetics and the like in addition to the compounds described herein.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically.

The pharmaceutical compositions herein described may comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of active ingredient effective in modulating hair growth of the subject being treated.

Determination of a therapeutically effective amount is well within the capability of those skilled in the art, especially in light of the detailed disclosure provided herein.

Toxicity and therapeutic efficacy of the compounds described herein can be determined by standard pharmaceutical procedures in cell cultures or experimental animals, e.g., by determining the IC₅₀ and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Depending on the severity and responsiveness of the condition to be treated, dosing can also be a single administration of a slow release composition using for example skin patches, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The present invention can be used to treat any one of a plurality of diseases, disorders or conditions associated with modulation of hair growth.

A skin absorption enhancer can be used in a composition of the present invention. Skin absorption enhancer include, for example, khellin, methyl nicotinate, MSM-Decy methyl sulfoxide, diethylene glycol, citric acid, pyruvic acid, phenoxyethanol, transcutol, GEMTEK surfactant, phosphatidyl choline, MCT oil and water.

The following Table 3 provides a range of concentrations of ingredients that may be used in the skin absorption enhancer.

TABLE 3

| SKIN ABSORTION ENHANCER | Weight % |
|---|---|
| Khellin | 0-10 |
| Methyl nicotinate | 0-20 |
| Decy methyl sulfoxide | 0-60 |
| Diethylene glycol | 0-90 |
| Citric acid | 0-45 |
| Pyruvic acid | 0-45 |
| Phenoxyethanol | 0-85 |
| Transcutol | 0-90 |
| GEMTEK surfactant | 0-20 |
| Phosphatidyl choline | 0-10 |
| MCT oil | 0-30 |
| Water | 0-80 |

The above ingredients are shown in weight percent, and are available from commercial suppliers such as Brooks, Sigma (St. Louis, Mo.) and Aldrich (Milwaukee, Wis.).

The following Table 4 provides a preferred formulation of the skin absorption enhancer.

TABLE 4

| SKIN ABSORTION ENHANCER | Weight % |
|---|---|
| Khellin | 0.1 |
| Methyl nicotinate | 0.2 |
| MSM-Decy methyl sulfoxide | 2 |
| Diethylene glycol | 4 |
| Citric acid | 4 |
| Pyruvic acid | 2 |
| Phenoxyethanol | 6 |
| Transcutol | 4.7 |
| GEMTEK surfactant | 0.25 |
| Phosphatidyl choline | 0.1 |
| MCT oil | 2 |
| Water | 74.65 |

The above ingredients are shown in weight percent, and are available from commercial suppliers such as Brooks, Sigma (St. Louis, Mo.) and Aldrich (Milwaukee, Wis.).

In the method of the present invention, for modulating hair growth, the following steps are performed preferably in the order noted: (i) cleansing the scalp or other body portion treated with a cleansing agent; (ii) optionally, treating the cleansed scalp or body portion with a keratin solvent system; (iii) optionally, applying a topical anesthetic; (iv) optionally, applying an acid peel solution; (v) optionally, applying a hyperactive urea gel formula and (vi) applying a hair growth modulating composition.

When the hair growth modulating composition includes a hair growth inducer, treatment can be applied to individuals with, for example, alopecia androgenetica, alopecia totalis, alopecia universalis and alopecia greata.

When the hair growth modulating composition includes a hair growth inhibitor, treatment can be applied to individuals with, for example, excessive hair growth, such as in hirsutism or for cosmetic purposes.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Demonstration of the Role of P-Cadherin in Hair Follicle Morphogenesis

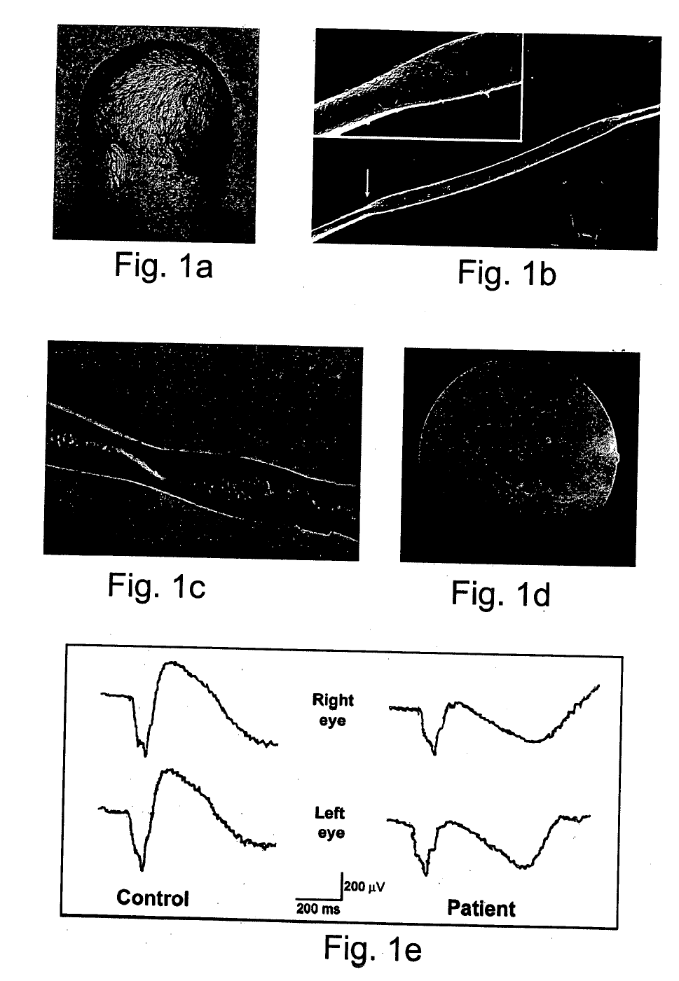

Four large consanguineous HJMD families with 11 affected individuals were selected for this study. All families originated from a small region of Northern Israel and belonged to the Druze population, a religious minority of Muslim origin, living in mountainous areas of the Middle East as a closed society almost from its inception in Cairo around 1017 A. C. (Qumsiyeh, M. B., Dasouki M. J. & Teebi, A. S. In: Genetic disorders among Arab populations, Teebi, A. S. & Farag, T. I. eds., p.232, Oxford University Press, Oxford (1997)). Affected individuals were born with normal-appearing hair but developed alopecia of the scalp at about 3 months of age. During puberty, however, partial regrowth of short and sparse hair occurred (FIG. 1a). Histological examination of scalp skin biopsies showed normal findings except for a reduced ratio of terminal vs. vellus hair follicles while distinct structural aberrations of the hair shafts were evident by light and scanning electron microscopic examinations (FIG. 1b-c). Between the age of 3 and 21 years, affected individuals developed progressive macular degeneration with slight peripheral retinal dystrophy (FIG. 1d). Electrophysiological evaluation of the visual system disclosed anomalies consistent with impaired macular function (FIG. 1e).

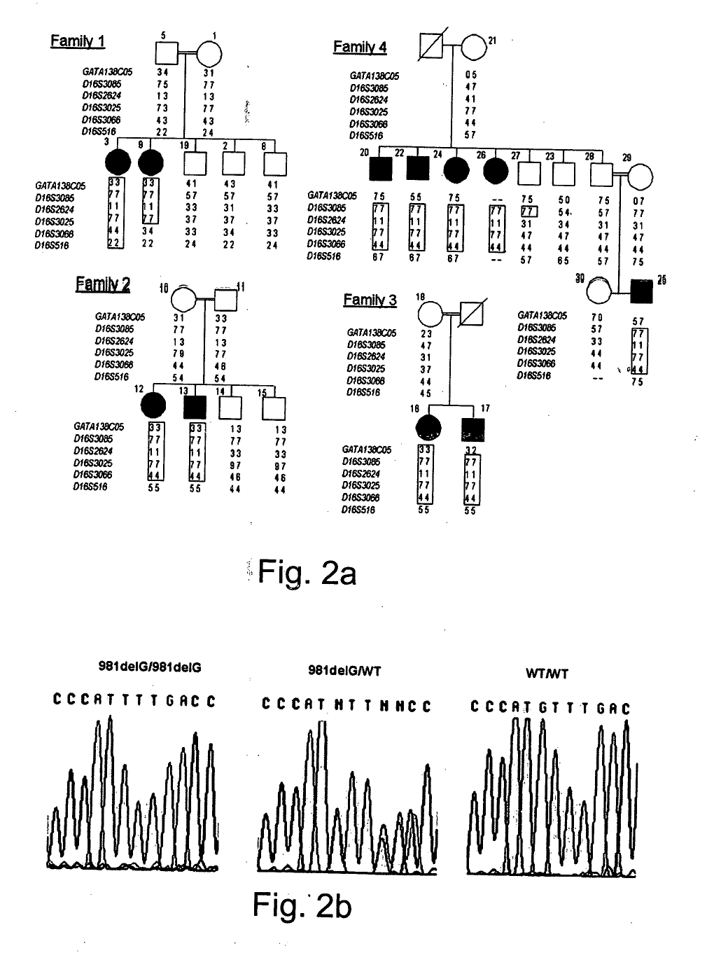

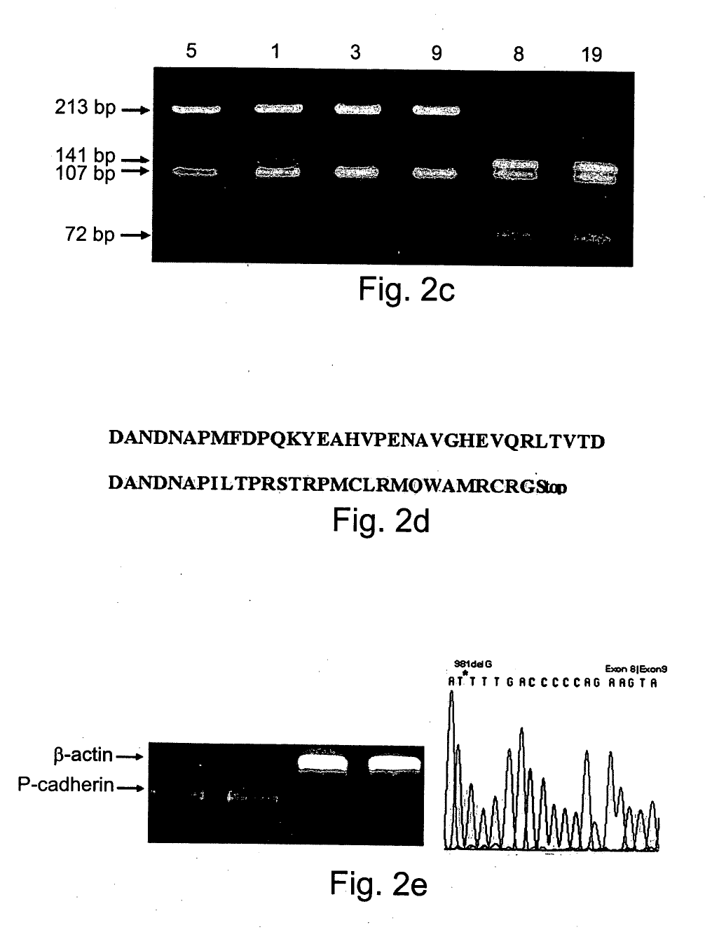

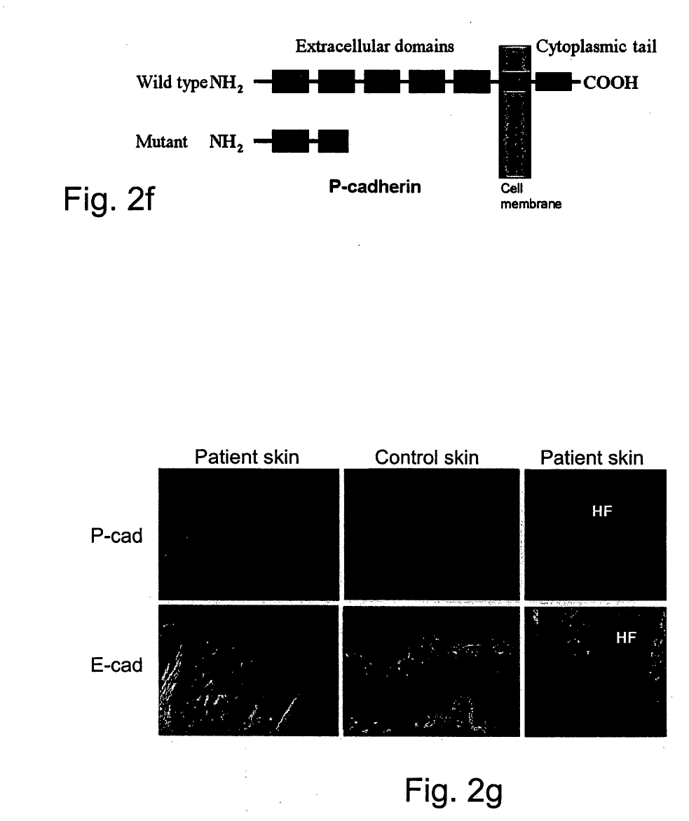

With informed consent of all participants, DNA was obtained from peripheral blood samples for molecular studies. To map the HJMD gene, a genome wide scan was performed by genotyping 202 fluorescently-labeled microsatellite markers (Research Genetics). Consanguinity of the families enabled to apply homozygosity mapping to identify a 20 cM segment on chromosome 16q22.1 identical by descent in affected individuals of families 1-3. Subsequent haplotype analysis and multipoint linkage analysis (HOMOZ software, Kruglyak, L., Daly, M. J. & Lander, E. S. Am. J. Hum. Genet. 56, 519-527 (1995)) using 5 additional polymorphic markers in all members of the 4 families further refined the disease gene locus to a 5 cM interval flanked by D16S3085 and D16S3066 (FIG. 2a) with a maximum 10d score of 10.4 at marker D16S3025.

Three contigs were identified in the unfinished High Throughput Genomic Sequences (htgs) database that contained at least one of the 4 microsatellite markers flanking or located within the HJMD critical interval. Together these contigs harbored at least 45 different genes, including CDH3 encoding P-cadherin. Following are the Genbank accession numbers of contigs within the critical disease interval: NT_{—}010478; NT_{—}024792; NT_{—}010556; CDH3 cDNA: NM_{—}001793.

Classical cadherins are thought to be involved in the regulation of hair (Fukumi, F. et al. Microsc. Res. Tech. 38, 343-352 (1997); Muller-Rover, S. et al. Exp. Dermatol. 8, 237-246 (1999)) as well as retinal (Riehl, R. et al. Neuron 17, 837-848 (1996)) development. CDH3 spans 55.45 kb, comprises 16 exons and is part of a cluster of cadherin genes located on 16q (Kremmidiotis, G., Baker, E., Crawford, J., Eyre, H. J., Nahmias, J. & Callen, D. F. Genomics 49, 467-471 (1998)). The organization of P-cadherin conforms to the general structure of classical cadherins with 5 extracellular domains, a transmembrane region and a short intracellular tail (Yagi, T. & Takeishi, M. Genes Dev. 14, 1169-1180 (2000)) (FIG. 2f).

The entire coding region of CDH3 was PCR-amplified and directly sequenced, including exon-intron boundaries, in one affected individual. The following primer pairs (presented in a 5′ to 3′ orientation) were employed:

| | | |
|---|---|---|
| CDH3/16F | CTTGGAGATGCTCTGTGGC | (SEQ ID NO:46) |
| CDH3/16R | GCACTTGCTGTCTGCTGGTC | (SEQ ID NO:47) |
| CDH3/15F | CATGCTTGTTCTCCTGTGTG | (SEQ ID NO:48) |
| CDH3/15R | CTGTGACATCATCTGTCTTG | (SEQ ID NO:49) |
| CDH3/14F | CAAAGAGACTACAGCAATGGAC | (SEQ ID NO:50) |
| CDH3/14R | CTGAGTGAGGACATCTGCAG | (SEQ ID NO:51) |
| CDH3/13F | CTGGGTGACAGAGTGAGAC | (SEQ ID NO:52) |
| CDH3/13R | CTTCATGGTGTACTCAGATC | (SEQ ID NO:53) |
| CDH3/12F | GGTTCTAGAGGAGATCATTGTC | (SEQ ID NO:54) |
| CDH3/12R | GTCTTGAGAGGTGAGAGCTG | (SEQ ID NO:55) |
| CDH3/11F | GCATGAGCCACTGCATCCAG | (SEQ ID NO:56) |
| CDH3/11R | GCCCTGAATGATGACATCAG | (SEQ ID NO:57) |
| CDH3/10F | CAATCTCTATGGTAATCAGAAC | (SEQ ID NO:58) |
| CDH3/10R | CATCTCAACTGTCCTGCACAG | (SEQ ID NO:59) |
| CDH3/9F | CAGTGACTCTTACCTATTTATG | (SEQ ID NO:60) |
| CDH3/9R | CATCCTGCCGCTGTGTATAC | (SEQ ID NO:61) |
| CDH3/8F | CAGCCATAGTGCTGAGACTG | (SEQ ID NO:62) |
| CDH3/8R | CACCCATGAGCCAGTGCTTC | (SEQ ID NO:63) |
| CDH3/7F | GCTTCTGCTCTCAGAGTCAG | (SEQ ID NO:64) |
| CDH3/7R | GTAGACAGGGCTGGAGTTG | (SEQ ID NO:65) |
| CDH3/5 + 6F | CAGAGCTCTGCTCTAGGATC | (SEQ ID NO:66) |
| CDH3/5 + 6R | CTGTTCAGTGAGCAGATTCTC | (SEQ ID NO:67) |
| CDH3/4F | CAGTAGCAAGAAATCTCATGC | (SEQ ID NO:68) |
| CDH3/4R | CAATAGGCTCATCTAGGTCTC | (SEQ ID NO:69) |
| CDH3/3F | GACTAACACTACCTCCTCTG | (SEQ ID NO:70) |
| CDH3/3R | GTCCATGAATGTCTATGATC | (SEQ ID NO:71) |
| CDH3/2F | GATGTCATAGGCGCTCTGCTG | (SEQ ID NO:72) |
| CDH3/2R | GTCGCGGCAGCTGCTTCAC | (SEQ ID NO:73) |
| CDH3/1F | GCAGAGAGTGAAGGAGGCTG | (SEQ ID NO:74) |
| CDH3/1R | GTACTGAGGAGGCTGAGGAG | (SEQ ID NO:75) |

PCR conditions were optimized for each primer pair.

A homozygous deletion of a guanine nucleotide was identified in exon 8 at position 981 from the translation start site (ATG) of CDH3 (FIG. 2b). The 981delG mutation abolishes a recognition site for NlaIII (FIG. 2c) and is predicted to result in a frameshift that introduces a premature termination codon 23 residues downstream of the mutation site (FIG. 2d). Using direct DNA sequencing and restriction fragment analysis, it was determined that all affected individuals were homozygous for the 981delG mutation, and that their parents were carriers of the mutant allele. In contrast, the mutation was not found in a pool of 248 chromosomes of healthy unrelated Druze, Arab-Israeli and Caucasians individuals, excluding the possibility that the 981delG mutation represents a non-consequential polymorphism. Affected individuals also shared an ancestral haplotype for markers D16S3085, D16S3025 and D16S2624 (FIG. 2a), although a genealogical relationship could only be defined between families 2 and 3. These results strongly suggest a founder effect for 981delG in the Druze population.

To study the consequences of the 981delG mutation, a skin biopsy was obtained from a homozygous HJMD patient. The level of CDH3 mRNA expression determined by semi-quantitative RT-PCR was equivalent to that of a normal control sample suggesting either absence of nonsense-mediated RNA decay (Frischmeyer, P. A. & Dietz, H. C. Hum. Mol. Genet. 8, 1893-1900 (1999)) or RNA decay with compensatory overexpression of CDH3 (FIG. 2e). Direct sequence analysis of RT-PCR products confirmed the presence of the CDH3 mutation in the patient's cDNA and did not provide evidence for exon skipping (FIG. 2e). The 981delG mutation is predicted to result in translation of a truncated protein lacking its cytoplasmic tail and 3 out of 5 extracellular domains (FIG. 2f). P-cadherin membranal expression was assessed by immunofluorescence staining and shown to be markedly reduced in patient skin biopsies (FIG. 2g), suggesting either protein degradation or loss of antigenic epitope. These results indicate that HJMD is caused by the loss of P-cadherin function due to a frameshift mutation in CDH3. P-cadherin expression has been demonstrated in the retinal pigment epithelium (Burke, J. M., Cao, F., Irving, P. E. & Skumatz, C. M. Invest. Ophthalmol. Vis. Sci. 40, 2963-2970 (1999)), although the exact role of P-cadherin in retina development remains elusive. Interestingly, two other forms of retinal dystrophy (Usher syndromes type 1D and 1F) have been shown to result from mutations in unrelated cadherin genes (Ahmed, Z. M. et al. Am. J. Hum. Genet. 69, 25-34 (2001); Bolz, H. et al. Nature Genet. 27, 108-112 (2001)). In the hair follicle, P-cadherin (but not E-cadherin) is expressed in a subset of epithelial cells involved in hair shaft growth regulation (Muller-Rover, S. et al. Exp. Dermatol. 8, 237-246 (1999)), an observation which may help understanding the peculiar HJMD phenotype. In contrast, most other epithelia co-express both P-cadherin and E-cadherin, and the latter might be able to compensate, at least in part, for P-cadherin deficiency in epidermal cells (Lewis, J. E., Jensen, P. J. & Wheelock, M. J J. Invest. Dermatol. 102, 870-877 (1994)), thus explaining the absence of skin phenotype in HJMD patients. Some form of functional redundancy may also explain the characteristic regrowth of hair in HJMD patients during puberty. Indeed gene expression of various cadherins and cadherin-related proteins, such as E-cadherin (Chen, G. T., Getsios, S. & MacCalman, C. D. Endocrine 9, 263-267 (1998))¹⁶ and β-catenin (Monks, D. A., Getsios, S., MacCalman, C. D. & Watson, N. V. Proc. Natl. Acad. Sci. U.S.A. 98, 1312-1316 (2001)), has been shown to be controlled by sex hormones. It is of interest to note that loss of P-cadherin in mice does not result in obvious hair or ophthalmological abnormalities (Radice, G. L. et al. J. Cell Biol. 139, 1025-1032 (1997)). Such phenotypic discrepancies between mice and humans carrying mutations in orthologous genes are not uncommon: mutations in another cadherin gene, PCDH15, cause retinitis pigmentosa in humans but not in mice (Ahmed, Z. M. et al. Am. J. Hum. Genet. 69, 25-34 (2001)), and humans, but not mice, carrying recessive mutations in GJB3 display severe deafness (Plum, A. et al. Dev. Biol. 231, 334-347 (2001)).

Classical cadherins maintain cell-cell adhesion at adherens junctions through Ca⁺²-dependant homophilic interactions (Yagi, T. & Takeishi, M. Genes Dev. 14, 1169-1180 (2000)). β-catenin physically links the actin cytoskeleton to the cytoplasmic tail of P-cadherin (Yagi, T. & Takeishi, M. Genes Dev. 14, 1169-1180 (2000)), which is truncated as a result of the 981delG mutation. Since β-catenin was shown to control hair follicle mophogenesis (Huelsken, J., Vogel, R., Erdmann, B., Cotsarelis, G. & Birchmeier, W. Cell 105, 533-545 (2001)) and since constitutive expression of the β-catenin gene in mice leads to exuberant hair growth (Gat, U., DasGupta, R., Degenstein, L. & Fuchs, E. Cell 95, 605-614 (1998)), abnormal interactions between β-catenin and non-functional P-cadherin might play a pivotal role in the pathogenesis of HJMD.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of identifying a hair growth modulator comprising:
identifying a P-cadherin modulator; and
testing whether said P-cadherin modulator is functional as a hair growth modulator.

2. The method of claim 1, wherein said P-cadherin modulator is an antisense oligonucleotide capable of specifically binding to P-cadherin gene, pre-messenger RNA or messenger RNA under physiological conditions.

3. The method of claim 1, wherein said P-cadherin modulator is an antisense construct encoding an antisense transcript capable of specifically binding to P-cadherin gene, pre-messenger RNA or messenger RNA under physiological conditions.

4. The method of claim 1, wherein said P-cadherin modulator is a polynucleotide capable of directing P-cadherin expression in hair follicle cells.

5. The method of claim 1, wherein said P-cadherin modulator is an anti-P-cadherin antibody.

6. The method of claim 1, wherein said P-cadherin modulator is an a small molecular weight organic compound.

7. The method of claim 1, wherein said P-cadherin modulator is a peptide.

8. A hair growth modulator identified by the method of claim 1.

9. A method of modulating hair growth comprising administering to a subject in need a therapeutically effective amount of the hair growth modulator of claim 8.

10. A method of identifying a hair growth modulator comprising:
identifying a molecule being capable of specifically binding to P-cadherin; and
testing whether said molecule is functional as a hair growth modulator.

11. The method of claim 10, wherein said molecule is an anti-P-cadherin antibody.

12. The method of claim 10, wherein said molecule is an a small molecular weight organic compound.

13. The method of claim 10, wherein said molecule is a peptide.

14. A hair growth modulator identified by the method of claim 10.

15. A method of modulating hair growth comprising administering to a subject in need a therapeutically effective amount of the hair growth modulator of claim 14.

16. The method of claim 10, wherein identifying said molecule being capable of specifically binding to P-cadherin is by a two hybrid system.

17. A method of identifying a hair growth inhibitor comprising:
identifying a P-cadherin inhibitor; and
testing whether said P-cadherin inhibitor is functional as a hair growth inhibitor.

18. The method of claim 17, wherein said P-cadherin inhibitor is an antisense oligonucleotide capable of specifically binding to P-cadherin gene, pre-messenger RNA or messenger RNA under physiological conditions.

19. The method of claim 17, wherein said P-cadherin inhibitor is an antisense construct encoding an antisense transcript capable of specifically binding to P-cadherin gene, pre-messenger RNA or messenger RNA under physiological conditions.

20. The method of claim 17, wherein said P-cadherin inhibitor is an anti-P-cadherin antibody.

21. The method of claim 17, wherein said P-cadherin inhibitor is an a small molecular weight organic compound.

22. The method of claim 17, wherein said P-cadherin inhibitor is a peptide.

23. A hair growth inhibitor identified by the method of claim 17.

24. A method of inhibiting hair growth comprising administering to a subject in need a therapeutically effective amount of the hair growth inhibitor of claim 23.

25. A method of identifying a hair growth inhibitor comprising:
identifying a molecule being capable of specifically binding to P-cadherin; and
testing whether said molecule is functional as a hair growth inhibitor.

26. The method of claim 25, wherein said molecule is an anti-P-cadherin antibody.

27. The method of claim 25, wherein said molecule is an a small molecular weight organic compound.

28. The method of claim 25, wherein said molecule is a peptide.

29. A hair growth inhibitor identified by the method of claim 25.

30. A method of inhibiting hair growth comprising administering to a subject in need a therapeutically effective amount of the hair growth inhibitor of claim 29.

31. The method of claim 26, wherein identifying said molecule being capable of specifically binding to P-cadherin is by a two hybrid system.

32. A method of identifying a hair growth inducer comprising:
identifying a P-cadherin inducer; and
testing whether said P-cadherin inducer is functional as a hair growth inducer.

33. The method of claim 32, wherein said P-cadherin inducer is a polynucleotide capable of directing P-cadherin expression in hair follicle cells.

34. The method of claim 32, wherein said P-cadherin inducer is an a small molecular weight organic compound.

35. The method of claim 32, wherein said P-cadherin inducer is a peptide.

36. A hair growth inducer identified by the method of claim 32.

37. A method of inducing hair growth comprising administering to a subject in need a therapeutically effective amount of the hair growth inducer of claim 36.

38. A method of identifying a hair growth inducer comprising:
identifying a molecule being capable of specifically binding to P-cadherin; and
testing whether said molecule is functional as a hair growth inducer.

39. The method of claim 38, wherein said molecule is an anti-P-cadherin antibody.

40. The method of claim 38, wherein said molecule is an a small molecular weight organic compound.

41. The method of claim 38, wherein said molecule is a peptide.

42. A hair growth inducer identified by the method of claim 38.

43. A method of inducing hair growth comprising administering to a subject in need a therapeutically effective amount of the hair growth inducer of claim 42.

44. The method of claim 39, wherein identifying said molecule being capable of specifically binding to P-cadherin is by a two hybrid system.

45. A method of modulating hair growth, the method comprising administering to a subject in need a therapeutically effective amount of a P-cadherin modulator functional as a hair growth modulator.

46. The method of claim 45, wherein said P-cadherin modulator is an antisense oligonucleotide capable of specifically binding to P-cadherin gene, pre-messenger RNA or messenger RNA under physiological conditions.

47. The method of claim 45, wherein said P-cadherin modulator is an antisense construct encoding an antisense transcript capable of specifically binding to P-cadherin gene, pre-messenger RNA or messenger RNA under physiological conditions.

48. The method of claim 45, wherein said P-cadherin modulator is a polynucleotide capable of directing P-cadherin expression in hair follicle cells.

49. The method of claim 45, wherein said P-cadherin modulator is an anti-P-cadherin antibody.

50. The method of claim 45, wherein said P-cadherin modulator is an a small molecular weight organic compound.

51. The method of claim 45, wherein said P-cadherin modulator is a peptide.

52. The method of claim 45, further comprising co-administering to the subject a therapeutically effective amount of an additional hair growth modulator.

53. A method of inhibiting hair growth, the method comprising administering to a subject in need a therapeutically effective amount of a P-cadherin inhibitor functional as a hair growth inhibitor.

54. The method of claim 53, wherein said P-cadherin inhibitor is an antisense oligonucleotide capable of specifically binding to P-cadherin gene, pre-messenger RNA or messenger RNA under physiological conditions.

55. The method of claim 53, wherein said P-cadherin inhibitor is an antisense construct encoding an antisense transcript capable of specifically binding to P-cadherin gene, pre-messenger RNA or messenger RNA under physiological conditions.

56. The method of claim 53, wherein said P-cadherin inhibitor is an anti-P-cadherin antibody.

57. The method of claim 53, wherein said P-cadherin inhibitor is an a small molecular weight organic compound.

58. The method of claim 53 wherein said P-cadherin inhibitor is a peptide.

59. The method of claim 53, further comprising co-administering to the subject a therapeutically effective amount of an additional hair growth inhibitor.

60. A method of inducing hair growth, the method comprising administering to a subject in need a therapeutically effective amount of a P-cadherin inducer functional as a hair growth inducer.

61. The method of claim 60, wherein said P-cadherin inducer is a polynucleotide capable of directing P-cadherin expression in hair follicle cells.

62. The method of claim 60, wherein said P-cadherin inducer is an a small molecular weight organic compound.

63. The method of claim 60, wherein said P-cadherin inducer is a peptide.

64. The method of claim 60, further comprising co-administering to the subject a therapeutically effective amount of an additional hair growth inducer.

65. A pharmaceutical composition for modulating hair growth, the pharmaceutical composition comprising, as an active ingredient, a therapeutically effective amount of a P-cadherin modulator functional as a hair growth modulator.

66. The pharmaceutical composition for claim 65, wherein said P-cadherin modulator is an antisense oligonucleotide capable of specifically binding to P-cadherin gene, pre-messenger RNA or messenger RNA under physiological conditions.

67. The pharmaceutical composition for claim 65, wherein said P-cadherin modulator is an antisense oligonucleotide capable of specifically binding to P-cadherin gene, pre-messenger RNA or messenger RNA under physiological conditions.

68. The pharmaceutical composition for claim 65, wherein said P-cadherin modulator is a polynucleotide capable of directing P-cadherin expression in hair follicle cells.

69. The pharmaceutical composition for claim 65, wherein said P-cadherin modulator is an anti-P-cadherin antibody.

70. The pharmaceutical composition for claim 65, wherein said P-cadherin modulator is an a small molecular weight organic compound.

71. The pharmaceutical composition for claim 65, wherein said P-cadherin modulator is a peptide.

72. The pharmaceutical composition for claim 65, further comprising, as an additional active ingredient, a therapeutically effective amount of an additional hair growth modulator.

73. A pharmaceutical composition for inhibiting hair growth, the pharmaceutical composition comprising, as an active ingredient, a therapeutically effective amount of a P-cadherin inhibitor functional as a hair growth inhibitor.

74. The pharmaceutical composition for claim 73, wherein said P-cadherin inhibitor is an antisense oligonucleotide capable of specifically binding to P-cadherin gene, pre-messenger RNA or messenger RNA under physiological conditions.

75. The pharmaceutical composition for claim 73, wherein said P-cadherin inhibitor is an antisense construct encoding an antisense transcript capable of specifically binding to P-cadherin gene, pre-messenger RNA or messenger RNA under physiological conditions.

76. The pharmaceutical composition for claim 73, wherein said P-cadherin inhibitor is an anti-P-cadherin antibody.

77. The pharmaceutical composition for claim 73, wherein said P-cadherin inhibitor is an a small molecular weight organic compound.

78. The pharmaceutical composition for claim 73, wherein said P-cadherin inhibitor is a peptide.

79. The pharmaceutical composition for claim 73, further comprising, as an additional active ingredient, a therapeutically effective amount of an additional hair growth inhibitor.

80. A pharmaceutical composition for inducing hair growth, the pharmaceutical composition comprising, as an active ingredient, a therapeutically effective amount of a P-cadherin inducer functional as a hair growth inducer.

81. The pharmaceutical composition for claim 80, wherein said P-cadherin inducer is a polynucleotide capable of directing P-cadherin expression in hair follicle cells.

82. The pharmaceutical composition for claim 80, wherein said P-cadherin inducer is an a small molecular weight organic compound.

83. The pharmaceutical composition for claim 80, wherein said P-cadherin inducer is a peptide.

84. The pharmaceutical composition for claim 80, further comprising, as an additional active ingredient, a therapeutically effective amount of an additional hair growth inducer.


SEQUENCE LISTING (75 sequences)

SEQ ID NO: 1 - protein, 790 aa, Homo sapiens
MRTYRYFLLLFWVGQPYPTLSTPLSKRTSGFPAKKRALELSGNSKNELNRSKRSWMWNQFFLLEEYTGSDYQYVGKLHSDQDRGDGSLKYILSGDGAGDLFIINENTGDIQATKRLDREEKPVYILRAQAINRRTGRPVEPESEFIIKIHDINDNEPIFTKEVYTATVPEMSDVGTFVVQVTATDADDPTYGNSAKVVYSILQGQPYFSVESETGIIKTALLNMDRENREQYQVVIQAKDMGGQMGGLSGTTTVNITLTDVNDNPPRFPQSTYQFKTPESSPPGTPIGRIKASDADVGENAEIEYSITDGEGLDMFDVITDQETQEGIITVKKLLDFEKKKVYTLKVEASNPYVEPRFLYLGPFKDSATVRIVVEDVDEPPVFSKLAYILQIREDAQINTTIGSVTAQDPDAARNPVKYSVDRHTDMDRIFNIDSGNGSIFTSKLLDRETLLWHNITVIATEINNPKQSSRVPLYIKVLDVNDNAPEFAEFYETFVCEKAKADQLIQTLHAVDKDDPYSGHQFSFSLAPEAASGSNFTIQDNKDNTAGILTRKNGYNRHEMSTYLLPVVISDNDYPVQSSTGTVTVRVCACDHHGNMQSCHAEALIHPTGLSTGALVAILLCIVILLVTVVLFAALRRQRKKEPLIISKEDIRDNIVSYNDEGGGEEDTQAFDIGTLRNPEAIEDNKLRRDIVPEALFLPRRTPTARDNTDVRDFINQRLKENDTDPTAPPYDSLATYAYEGTGSVADSLSSLESVTTDADQDYDYLSDWGPRFKKLADMYGGVDSDKDS

SEQ ID NO: 2 - protein, 794 aa, Homo sapiens [sequence omitted]

SEQ ID NO: 3 - protein, 799 aa, Homo sapiens
MPERLAEMLLDLWTPLIILWITLPPCIYMAPMNQSQVLMSGSPLELNSLGEEQRILNRSKRGWVWNQMFVLEEFSGPEPILVGRLHTDLDPGSKKIKYILSGDGAGTIFQINDVTGDIHAIKRLDREEKAEYTLTAQAVDWETSKPLEPPSEFIIKVQDINDNAPEFLNGPYHATVPEMSILGTSVTNVTATDADDPVYGNSAKLVYSILEGQPYFSIEPETAIIKTALPNMDREAKEEYLVVIQAKDMGGHSGGLSGTTTLTVTLTDVNDNPPKFAQSLYHFSVPEDVVLGTAIGRVKANDQDIGENAQSSYDIIDGDGTALFEITSDAQAQDGIIRLRKPLDFETKKSYTLKVEAANVHIDPRFSGRGPFKDTATVKIVVEDADEPPVFSSPTYLLEVHENAALNSVIGQVTARDPDITSSPIRFSIDRHTDLERQFNINADDGKITLATPLDRELSVWHNITIIATEIRNHSQISRVPVAIKVLDVNDNAPEFASEYEAFLCENGKPGQVIQTVSAMDKDDPKNGHYFLYSLLPEMVNNPNFTIKKNEDNSLSILAKHNGFNRQKQEVYLLPIIISDSGNPPLSSTSTLTIRVCGCSNDGVVQSCNVEAYVLPIGLSMGALIAILACIILLLVIVVLFVTLRRHKNEPLIIKDDEDVRENIIRYDDEGGGEEDTEAFDIATLQNPDGINGFLPRKDIKPDLQFMPRQGLAPVPNGVDVDEFINVRLHEADNDPTAPPYDSIQIYGYEGRGSVAGSLSSLESTTSDSDQNFDYLSDWGPRFKRLGELYSVGESDKET

SEQ ID NO: 4 - protein, 796 aa, Homo sapiens
MKENYCLQAALVCLGMLCHSHAFAPERRGHLRPSFHGHHEKGKEGQVLQRSKRGWVWNQFFVIEEYTGPDPVLVGRLHSDIDSGDGNIKYILSGEGAGTIFVIDDKSGNIHATKTLDREERAQYTLMAQAVDRDTNRPLEPPSEFIVKVQDINDNPPEFLHETYHANVPERSNVGTSVIQVTASDADDPTYGNSAKLVYSILEGQPYFSVEAQTGIIRTALPNMDREAKEEYHVVIQAKDMGGHMGGLSGTTKVTITLTDVNDNPPKFPQRLYQMSVSEAAVPGEEVGRVKAKDPDIGENGLVTYNIVDGDGMESFEITTDYETQEGVIKLKKPVDFETERAYSLKVEAANVHIDPKFISNGPFKDTVTVKISVEDADEPPMFLAPSYIHEVQENAAAGTVVGRVHAKDPDAANSPIRYSIDRHTDLDRFFTINPEDGFIKTTKPLDREETAWLNITVFAAEIHNRHQEAQVPVAIRVLDVNDNAPKFAAPYEGFICESDQTKPLSNQPIVTISADDKDDTANGPRFIFSLPPEIIHNPNFTVRDNRDNTAGVYARRGGFSRQKQDLYLLPIVISDGGIPPMSSTNTLTIKVCGCDVNGALLSCNAEAYILNAGLSTGALIAILACIVILLVIVVLFVTLRRQKKEPLIVFEEEDVRENIITYDDEGGGEEDTEAFDIATLQNPDGINGFIPRKDIKPEYQYMPRPGLRPAPNSVDVDDFINTRIQEADNDPTAPPYDSIQIYGYEGRGSVAGSLSSLESATTDSDLDYDYLQNWGPRFKKLADLYGSKDTFDDDS

SEQ ID NO: 5 - protein, 784 aa, Homo sapiens
MQRLMMLLATSGACLGLLAVAAVAAAGANPAQRDTHSLLPTHRRQKRDWIWNQMHIDEEKNTSLPHHVGKIKSSVSRKNAKYLLKGEYVGKVFRVDAETGDVFAIERLDRENISEYHLTAVIVDKDTGENLETPSSFTIKVHDVNDNWPVFTHRLFNASVPESSAVGTSVISVTAVDADDPTVGDHASVMYQILKGKEYFAIDNSGRIITITKSLDREKQARYEIVVEARDAQGLRGDSGTATVLVTLQDINDNFPFFTQTKYTFVVPEDTRVGTSVGSLFVEDPDEPQNRMTKYSILRGDYQDAFTIETNPAHNEGIIKPMKPLDYEYIQQYSFIVEATDPTIDLRYMSPPAGNRAQVIINITDVDEPPIFQQPFYHFQLKENQKKPLIGTVLAMDPDAARHSIGYSIRRTSDKGQFFRVTKKGDIYNEKELDREVYPWYNLTVEAKELDSTGTPTGKESIVQVHIEVLDENDNAPEFAKPYQPKVCENAVHGQLVLQISAIDKDITPRNVKFKFTLNTENNFTLTDNHDNTANITVKYGQFDREHTKVHFLPVVISDNGMPSRTGTSTLTVAVCKCNEQGEFTFCEDMAAQVGVSIQAVVAILLCILTITVITLLIFLRRRLRKQARAHGKSVPEIHEQLVTYDEEGGGEMDTTSYDVSVLNSVRRGGAKPPRPALDARPSLYAQVQKPPRHAPGAHGGPGEMAAMIEVKKDEADHDGDGPPYDTLHIYGYEGSESIAESLSSLGTDSSDSDVDYDFLNDWGPRFKMLAELYGSDPREELLY

SEQ ID NO: 6 - protein, 829 aa, Homo sapiens [sequence omitted]

SEQ ID NO: 7 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 8 - protein, 900 aa, Homo sapiens
MCRIAGALRTLLPLLLALLQASVEASGEIALCKTGFPEDVYSAVLSKDVHEGQPLLNVKFSNCNGKRKVQYESSEPADFKVDEDGMVYAVRSFPLSSEHAKFLIYAQDKETQEKWQVAVKLSLKPTLTEESVKESAEVEEIVFPRQFSKHSGHLQRQKRDWVIPPINLPENSRGPFPQELVRIRSDRDKNLSLRYSVTGPGADQPPTGIFIINPISGQLSVTKPLDREQIARFHLRAHAVDINGNQVENPIDIVINVIDMNDNRPEFLHQVWNGTVPEGSKPGTYVMTVTAIDADDPNALNGMLRYRIVSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVYRISGGDPTGRFAIQTDPNSNDGLVTVVKPIDFETNRMFVLTVAAENQVPLAKGIQHPPQSTATVSVTVIDVNENPYFAPNPKIIRQEEGLHAGTMLTTFTAQDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITALDYDIDPNAGPFAFDLPLSPVTIKRNWTITRLNGDFAQLNLKIKFLEAGIYEVPIIITDSGNPPKSNISILRVKVCQCDSNGDCTDVDRIVGAGLGTGAIIAILLCIIILLILVLMFVVWMKRRDKERQAKQLLIDPEDDVRDNILKYDEEGGGEEDQDYDLSQLQQPDTVEPDAIKPVGIRRMDERPIHAEPQYPVRSAAPHPGDIGDFINEGLKAADNDPTAPPYDSLLVFDYEGSGSTAGSLSSLNSSSSGGEQDYDYLNDWGPRFKKLADM

SEQ ID NO: 9 - protein, 916 aa, Homo sapiens [sequence omitted]

SEQ ID NO: 10 - protein, 814 aa, Homo sapiens
MDAAFLLVLGLLAQSLCLSLGVPGWRRPTTLYPWRRAPALSRVRRAWVIPPISVSENHKRLPYPLVQIKSDKQQLGSVIYSIQGPGVDEEPRGVFSIDKFTGKVFLNAMLDREKTDRFRLRAFALDLGGSTLEDPTDLEIVVVDQNDNRPAFLQEAFTGRVLEGAVPGTYVTRAEATDADDPETDNAALRFSILQQGSPELFSIDELTGEIRTVQVGLDREVVAVYNLTLQVADMSGDGLTATASAIITLDDINDNAPEFTRDEFFMEAIEAVSGVDVGRLEVEDRDLPGSPNWVARFTILEGDPDGQFTIRTDPKTNEGVLSIVKALDYESCEHYELKVSVQNEAPLQAAALRAERGQAKVRVHVQDTNEPPVFQENPLRTSLAEGAPPGTLVATFSARDPDTEQLQRLSYSKDYDPEDWLQVDAATGRIQTQHVLSPASPFLKGGWYRAIVLAQDDASQPRTATGTLSIEILEVNDHAPVLAPPPPGSLCSEPHQGPGLLLGATDEDLPPHGAPFHFQLSPRLPELGRNWSLSQVNVSHARLRPRHQVPEGLHRLSLLLRDSGQPPQQREQPLNVTVCRCGKDGVCLPGAAALLAGGTGLSLGALVIVLASALLLLVLVLLVALRARFWKQSRGKGLLHGPQDDLRDNVLNYDEQGGGEEDQDAYDISQLRHPTALSLPLGPPPLRRDAPQGRLHPQPPRVLPTSPLDIADFINDGLEAADSDPSVPPYDTALIYDYEGDGSVAGTLSSILSSQGDEDQDYDYLRDWGPRFARLADMYGHPCGLEYGARWDHQAREGLSPGALLPRHRGRTA

SEQ ID NO: 11 - DNA, 3205 bp, Homo sapiens
aaaggggcaagagctgagcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatcgtgaccgaccagaatgaccacaagcccaagtttacccaggacaccttccgagggagtgtcttagagggagtcctaccaggtacttctgtgatgcaggtgacagccacggatgaggatgatgccatctacacctacaatggggtggttgcttactccatccatagccaagaaccaaaggacccacacgacctcatgttcaccattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaatgaccatggcccagtccctgagccccgtcagatcaccatctgcaaccaaagccctgtgcgccaggtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaagggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctcggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttcccagaaaaaaa

SEQ ID NO: 12 - DNA, 4758 bp, Homo sapiens
ggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgccggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgcctttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaattttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccataaaaaaaaaaaaa

SEQ ID NO: 13 - DNA, 4122 bp, Homo sapiens
tttgtcatcagctcgctctccattggcggggagcggagagcagcgaagaagggggtggggaggggaggggaagggaagggggtggaaactgcctggagccgtttctccgcgccgctgttggtgctgccgctgcctcctcctcctccgccgccgccgccgccgccgccgcctcctccggctcttcgctcggcccctctccgcctccatgtgccggatagcgggagcgctgcggaccctgctgccgctgctggcggccctgcttcaggcgtctgtagaggcttctggtgaaatcgcattatgcaagactggatttcctgaagatgtttacagtgcagtcttatcgaaggatgtgcatgaaggacagcctcttctcaatgtgaagtttagcaactgcaatggaaaaagaaaagtacaatatgagagcagtgagcctgcagattttaaggtggatgaagatggcatggtgtatgccgtgagaagctttccactctcttctgagcatgccaagttcctgatatatgcccaagacaaagagacccaggaaaagtggcaagtggcagtaaaattgagcctgaagccaaccttaactgaggagtcagtgaaggagtcagcagaagttgaagaaatagtgttcccaagacaattcagtaagcacagtggccacctacaaaggcagaagagagactgggtcatccctccaatcaacttgccagaaaactccaggggaccttttcctcaagagcttgtcaggatcaggtctgatagagataaaaacctttcactgcggtacagtgtaactgggccaggagctgaccagcctccaactggtatcttcattatcaaccccatctcgggtcagctgtcggtgacaaagcccctggatcgcgagcagatagcccggtttcatttgagggcacatgcagtagatattaatggaaatcaagtggagaaccccattgacattgtcatcaatgttattgacatgaatgacaacagacctgagttcttacaccaggtttggaatgggacagttcctgagggatcaaagcctggaacatatgtgatgaccgtaacagcaattgatgctgacgatcccaatgccctcaatgggatgttgaggtacagaatcgtgtctcaggctccaagcaccccttcacccaacatgtttacaatcaacaatgagactggtgacatcatcacagtggcagctggacttgatcgagaaaaagtgcaacagtatacgttaataattcaagctacagacatggaaggcaatcccacatatggcctttcaaacacagccacggccgtcatcacagtgacagatgtcaatgacaatcctccagagtttactgccatgacgttttatggtgaagttcctgagaacagggtagacatcatagtagctaatctaactgtgaccgataaggatcaaccccatacaccagcctggaacgcagtgtacagaatcagtggcggagatcctactggacggttcgccatccagaccgacccaaacagcaacgacgggttagtcaccgtggtcaaaccaatcgactttgaaacaaataggatgtttgtccttactgttgctgcagaaaatcaagtgccattagccaagggaattcagcacccgcctcagtcaactgcaaccgtgtctgttacagttattgacgtaaatgaaaacccttattttgcccccaatcctaagatcattcgccaagaagaagggcttcatgccggtaccatgttgacaacattcactgctcaggacccagatcgatatatgcagcaaaatattagatacactaaattatctgatcctgccaattggctaaaaatagatcctgtgaatggacaaataactacaattgctgttttggaccgagaatcaccaaatgtgaaaaacaatatatataatgctactttccttgcttctgacaatggaattcctcctatgagtggaacaggaacgctgcagatctatttacttgatattaatgacaatgcccctcaagtgttacctcaagaggcagagacttgcgaaactccagaccccaattcaattaatattacagcacttgattatgacattgatccaaatgctggaccatttgcttttgatcttcctttatctccagtgactattaagagaaattggaccatcactcggcttaatggtgattttgctcagcttaatttaaagataaaatttcttgaagctggtatctatgaagttcccatcataatcacagattcgggtaatcctcccaaatcaaatatttccatcctgcgcgtgaaggtttgccagtgtgactccaacggggactgcacagatgtggacaggattgtgggtgcggggcttggcaccggtgccatcattgccatcctgctctgcatcatcatcctgcttatccttgtgctgatgtttgtggtatggatgaaacgccgggataaagaacgccaggccaaacaacttttaattgatccagaagatgatgtaagagataatattttaaaatatgatgaagaaggtggaggagaagaagaccaggactatgacttgagccagctgcagcagcctgacactgtggagcctgatgccatcaagcctgtgggaatccgacgaatggatgaaagacccatccacgctgagccccagtatccggtccgatctgcagccccacaccctggagacattggggacttcattaatgagggccttaaagcggctgacaatgaccccacagctccaccatatgactccctgttagtgtttgactatgaaggcagtggctccactgctgggtccttgagctcccttaattcctcaagtagtggtggtgagcaggactatgattacctgaacgactgggggccacggttcaagaaacttgctgacatgtatggtggaggtgatgactgaacttcagggtgaacttggtttttggacaagtacaaacaatttcaactgatattcccaaaaagcattcagaagctaggctttaactttgtagtctactagcacagtgcttgctggaggctttggcataggctgcaaaccaatttgggctcagagggaatatcagtgatccatactgtttggaaaaacactgagctcagttacacttgaattttacagtacagaagcactgggattttatgtgcctttttgtacctttttcagattggaattagttttctgtttaaggctttaatggtactgatttctgaaacgataagtaaaagacaaaatattttgtggtgggagcagtaagttaaaccatgatatgcttcaacacgcttttgttacattgcatttgcttttattaaaatacaaaattaaacaaacaaaaaaactcatggagcgattttattatcttgggggatgagaccatgagattggaaaatgtacattacttctagttttagactttagtttgttttttttttttcactaaaatcttaaaacttactcagctggttgcaaataaagggagttttcatatcaccaatttgtagcaaaattgaattttttcataaactagaatgttagacacattttggtcttaatccatgtacacttttttatttctgtatttttccacttcactgtaaaaatagtatgtgtacataatgttttattggcatagtctatggagaagtgcagaaacttcagaacatgtgtatgtattatttggactatggattcaggttttttgcatgtttatatctttcgttatggataaagtatttacaaaacagtgacatttgattcaattgttgagctgtagttagaatactcaatttttaatttttttaatttttttattttttattttctttttggtttggggagggagaaaagttcttagcacaaatgttttacataatttgtaccaaaaaaaaaaaaaaggaaaggaaagaaaggggtggcctgacactggtggcactactaagtgtgtgttttttaaaaaaaaaaatggaaaaaaaaaagcttttaaactggagagacttctgacaacagctttgcctctgtattgtgtaccagaatataaatgatacacctctgaccccagcgttctgaataaaatgctaattttggaaaaaaaaaaaaaaaaa

SEQ ID NO: 14 - DNA, 3063 bp, Homo sapiens
cgccggcggggaagatgaccgcgggcgccggcgtgctccttctgctgctctcgctctccggcgcgctccgggcccataatgaggatcttacaactagagagacctgcaaggctgggttctctgaagatgattacacggcattaatctcccaaaatattctagaaggggaaaagctacttcaagtcaagttcagcagctgtgtggggaccaaggggacacaatatgagaccaacagcatggacttcaaagttggggcagatgggacagtcttcgccacccgggagctgcaggtcccctccgagcaggtggcgttcacggtgactgcatgggacagccagacagcagagaaatgggacgccgtggtgcggttgctggtggcccagacctcgtccccgcactctggacacaagccgcagaaaggaaagaaggtcgtggctctggacccctctccgcctccgaaggacaccctgctgccgtggccccagcaccagaacgccaacgggctgaggcggcgcaaacgggactgggtcatcccgcccatcaacgtgcccgagaactcgcgcgggcccttcccgcagcagctcgtgaggatccggtccgacaaagacaatgacatccccatccggtacagcatcacgggagtgggcgccgaccagccccccatggaggtcttcagcattgactccatgtccggccggatgtacgtcacaaggcccatggaccgggaggagcacgcctcttaccacctccgagcccacgctgtggacatgaatggcaacaaggtggagaaccccatcgacctgtacatctacgtcatcgacatgaatgacaaccgccctgagttcatcaaccaggtctacaacggctccgtggacgagggctccaagccaggcacctacgtgatgaccgtcacggccaacgatgctgacgacagcaccacggccaacgggatggtgcggtaccggatcgtgacccagaccccacagagcccgtcccagaatatgttcaccatcaacagcgagactggagatatcgtcacagtggcggctggcctggaccgagagaaagttcagcagtacacagtcatcgttcaggccacagatatggaaggaaatctcaactatggcctctcaaacacagccacagccatcatcacggtgacagatgtgaatgacaacccgccagaatttaccgccagcacgtttgcaggggaggtccccgaaaaccgcgtggagaccgtggtcgcaaacctcacggtgatggaccgagatcagccccactctccaaactggaatgccgtttaccgcatcatcagtggggatccatccgggcacttcagcgtccgcacagaccccgtaaccaacgagggcatggtcaccgtggtgaaggcagtcgactacgagctcaacagagctttcatgctgacagtgatggtgtccaaccaggcgcccctggccagcggaatccagatgtccttccagtccacggcaggggtgaccatctccatcatggacatcaacgaggctccctacttcccctcaaaccacaagctgatccgcctggaggagggcgtgccccccggcaccgtgctgaccacgttttcagctgtggaccctgaccggttcatgcagcaggctgtgagatactcaaagctgtcagacccagcgagctggctgcacatcaatgccaccaacggccagatcaccacggcggcagtgctggaccgtgagtccctctacaccaaaaacaacgtctacgaggccaccttcctggcagctgacaatgggatacccccggccagcggcaccgggaccctccagatctatctcattgacatcaacgacaacgcccctgagctgctgcccaaggaggcgcagatctgcgagaagcccaacctgaacgccatcaacatcacggcggccgacgctgacgtcgaccccaacatcggcccctacgtcttcgagctgccctttgtcccggcggccgtgcggaagaactggaccatcacccgcctgaacggtgactatgcccaactcagcttgcgcatcctgtacctggaggccgggatgtatgacgtccccatcatcgtcacagactctggaaaccctcccctgtccaacacgtccatcatcaaagtcaaggtgtgcccatgtgatgacaacggggactgcaccaccattggcgcagtggcagcggctggtctgggcaccggtgccatcgtggccatcctcatctgcatcctcatcctgctgaccatggtcctgctgtttgtcatgtggatgaagcggcgagagaaggagcgccacacgaagcagctgctcattgaccccgaggacgacgtccgcgacaacatcctcaagtatgacgaggaaggcggtggcgaggaggaccaggactacgacctcagccagctgcagcagccggaagccatggggcacgtgccaagcaaagcccctggcgtgcgtcgcgtggatgagcggccggtgggcgctgagccccagtacccgatcaggcccatggtgccgcacccaggcgacatcggtgacttcatcaatgagggactccgcgctgctgacaacgaccccacggcacccccctatgactccctgctggtcttcgactacgaggggagcggctccaccgcaggctccgtcagctccctgaactcatccagttccggggaccaagactacgattacctcaacgactgggggcccagattcaagaagctggcggacatgtatggaggtggtgaagaggattgactgacctcgcatcttcggaccgaagtgagagccgtgctcggacgccggaggagcaggactgagcagaggcggccggtcttcccgactccctgcggctgtgtccttagtgctgttaggaggccccccaatccccacgttgagctgtctagcatgagcacccacccccacagcgccctgcacccggccgctgcccagcaccgcgctggctggcactgaaggacagcaagaggcactctgtcttcacttgaatttcctagaacagaagcactgtttttaaaaaaaaaaaaaaaaaaagaag

SEQ ID NO: 15 - DNA, 2833 bp, Homo sapiens
acttgcgctgtcactcagcctggacgcgcttcttcgggtcgcgggtgcactccggcccggctcccgcctcggccccgatggacgccgcgttcctcctcgtcctcgggctgttggcccagagcctctgcctgtctttgggggttcctggatggaggaggcccaccaccctgtacccctggcgccgggcgcctgccctgagccgcgtgcggagggcctgggtcatccccccgatcagcgtatccgagaaccacaagcgtctcccctaccccctggttcagatcaagtcggacaagcagcagctgggcagcgtcatctacagcatccagggacccggcgtggatgaggagccccggggcgtcttctctatcgacaagttcacagggaaggtcttcctcaatgccatgctggaccgcgagaagactgatcgcttcaggctaagagcgtttgccctggacctgggaggatccaccctggaggaccccacggacctggagattgtagttgtggatcagaatgacaaccggccagccttcctgcaggaggcgttcactggccgcgtgctggagggtgcagtcccaggcacctatgtgaccagggcagaggccacagatgccgacgaccccgagacggacaacgcagcgctgcggttctccatcctgcagcagggcagccccgagctcttcagcatcgacgagctcacaggagagatccgcacagtgcaagtggggctggaccgcgaggtggtcgcggtgtacaatctgaccctgcaggtggcggacatgtctggagacggcctcacagccactgcctcagccatcatcacccttgatgacatcaatgacaatgcccccgagttcaccagggatgagttcttcatggaggccatagaggccgtcagcggagtggatgtgggacgcctggaagtggaggacagggacctgccaggctccccaaactgggtggccaggttcaccatcctggaaggcgaccccgatgggcagttcaccatccgcacggaccccaagaccaacgagggtgttctgtccattgtgaaggccctggactatgagagctgtgaacactacgaactcaaagtgtcggtgcagaatgaggccccgctgcaggcggctgcccttagggctgagcggggccaggccaaggtccgcgtgcatgtgcaggacaccaacgagccccccgtgttccaggagaacccacttcggaccagcctagcagagggggcacccccaggcactctggtggccaccttctctgcccgggaccctgacacagagcagctgcagaggctcagctactccaaggactacgacccggaagactggctgcaagtggacgcagccactggccggatccagacccagcacgtgctcagcccggcgtcccccttcctcaagggcggctggtacagagccatcgtcctggcccaggatgacgcctcccagccccgcaccgccaccggcaccctgtccatcgagatcctggaggtgaacgaccatgcacctgtgctggccccgccgccgccgggcagcctgtgcagcgagccacaccaaggcccaggcctcctcctgggcgccacggatgaggacctgcccccccacggggcccccttccacttccagctgagccccaggctcccagagctcggccggaactggagcctcagccaggtcaacgtgagccacgcgcgcctgcggccgcgacaccaggtccccgaaggcctgcaccgcctcagcctgctgctccgggactcggggcagccgccccagcagcgcgagcagcctctgaacgtgaccgtgtgccgctgcggcaaggacggcgtctgcctgccgggggccgcagcgctgctggcggggggcacaggcctcagcctgggcgcactggtcatcgtgctggccagcgccctcctgctgctggtgctggtcctgctcgtggcactccgggcgcggttctggaagcagtctcggggcaaggggctgctgcacggcccccaggacgaccttcgagacaatgtcctcaactacgatgagcaaggaggcggggaggaggaccaggacgcctacgacatcagccagctgcgtcacccgacagcgctgagcctgcctctgggaccgccgccacttcgcagagatgccccgcagggccgcctgcacccccagccaccccgagtgctgcccaccagccccctggacatcgccgacttcatcaatgatggcttggaggctgcagatagtgaccccagtgtgccgccttacgacacagccctcatctatgactacgagggtgacggctcggtggcggggacgctgagctccatcctgtccagccagggcgatgaggaccaggactacgactacctcagagactgggggccccgcttcgcccggctggcagacatgtatgggcacccgtgcgggttggagtacggggccagatgggaccaccaggccagggagggtctttctcctggggcactgctacccagacacagaggccggacagcctgaccctggggcgcaactggacatgccactccccggcctcgtggcagtgatggcccctgcagaggcagcctgaggtcaccgggcccgacccccctgggcctggggcagcctccttcctgtaggcgagggcccaagtctgggggcagaacctgagtgtggatggggcggccaggaagaggccccttcctgccggggtgggaagagtttctctccatcggccccatgcgggtcacctccctagtcccacctttgcctcctaccagtgaacctcatctttgtatgaaagacagcaacctcctgggtaaatctgaatg

SEQ ID NO: 16 - DNA, 4521 bp, Homo sapiens
acttcattcacttgcaaatcagtgtgtgcccacaagagccagctctcccgagcccgtaaccttcgcatcccaagagctgcagtttcagccgcgacagcaagaacggcagagccggcgaccgcggcggcggcggcggcggaggcaggagcagcctgggcgggtcgcagggtctccgcgggcgcaggaaggcgagcagagatatcctctgagagccaagcaaagaacattaaggaaggaaggaggaatgaggctggatacggtgcagtgaaaaaggcacttccaagagtggggcactcactacgcacagactcgacggtgccatcagcatgagaacttaccgctacttcttgctgctcttttgggtgggccagccctacccaactctctcaactccactatcaaagaggactagtggtttcccagcaaagaaaagggccctggagctctctggaaacagcaaaaatgagctgaaccgttcaaaaaggagctggatgtggaatcagttctttctcctggaggaatacacaggatccgattatcagtatgtgggcaagttacattcagaccaggatagaggagatggatcacttaaatatatcctttcaggagatggagcaggagatctcttcattattaatgaaaacacaggcgacatacaggccaccaagaggctggacagggaagaaaaacccgtttacatccttcgagctcaagctataaacagaaggacagggagacccgtggagcccgagtctgaattcatcatcaagatccatgacatcaatgacaatgaaccaatattcaccaaggaggtttacacagccactgtccctgaaatgtctgatgtcggtacatttgttgtccaagtcactgcgacggatgcagatgatccaacatatgggaacagtgctaaagttgtctacagtattctacagggacagccctatttttcagttgaatcagaaacaggtattatcaagacagctttgctcaacatggatcgagaaaacagggagcagtaccaagtggtgattcaagccaaggatatgggcggccagatgggaggattatctgggaccaccaccgtgaacatcacactgactgatgtcaacgacaaccctccccgattcccccagagtacataccagtttaaaactcctgaatcttctccaccggggacaccaattggcagaatcaaagccagcgacgctgatgtgggagaaaatgctgaaattgagtacagcatcacagacggtgaggggctggatatgtttgatgtcatcaccgaccaggaaacccaggaagggattataactgtcaaaaagctcttggactttgaaaagaagaaagtgtatacccttaaagtggaagcctccaatccttatgttgagccacgatttctctacttggggcctttcaaagattcagccacggttagaattgtggtggaggatgtagatgagccacctgtcttcagcaaactggcctacatcttacaaataagagaagatgctcagataaacaccacaataggctccgtcacagcccaagatccagatgctgccaggaatcctgtcaagtactctgtagatcgacacacagatatggacagaatattcaacattgattctggaaatggttcgatttttacatcgaaacttcttgaccgagaaacactgctatggcacaacattacagtgatagcaacagagatcaataatccaaagcaaagtagtcgagtacctctatatattaaagttctagatgtcaatgacaacgccccagaatttgctgagttctatgaaacttttgtctgtgaaaaagcaaaggcagatcagttgattcagaccctgcatgctgttgacaaggatgacccttatagtggacaccaattttcgttttccttggcccctgaagcagccagtggctcaaactttaccattcaagacaacaaagacaacacggcgggaatcttaactcggaaaaatggctataatagacacgagatgagcacctatctcttgcctgtggtcatttcagacaacgactacccagttcaaagcagcactgggacagtgactgtccgggtctgtgcatgtgaccaccacgggaacatgcaatcctgccatgcggaggcgctcatccaccccacgggactgagcacgggggctctggttgccatccttctgtgcatcgtgatcctactagtgacagtggtgctgtttgcagctctgaggcggcagcgaaaaaaagagcctttgatcatttccaaagaggacatcagagataacattgtcagttacaacgacgaaggtggtggagaggaggacacccaggcttttgatatcggcaccctgaggaatcctgaagccatagaggacaacaaattacgaagggacattgtgcccgaagcccttttcctaccccgacggactccaacagctcgcgacaacaccgatgtcagagatttcattaaccaaaggttaaaggaaaatgacacggaccccactgccccgccatacgactccttggccacttacgcctatgaaggcactggctccgtggcggattccctgagctcgctggagtcagtgaccacggatgcagatcaagactatgattaccttagtgactggggacctcgattcaaaaagcttgcagatatgtatggaggagtggacagtgacaaagactcctaatctgttgcctttttcattttccaatacgacactgaaatatgtgaagtggctatttctttatatttatccactactccgtgaaggcttctctgttctacccgttccaaaagccaatggctgcagtccgtgtggatccaatgttagagacttttttctagtacacttttatgagcttccaaggggcaaatttttattttttagtgcatccagttaaccaagtcagcccaacaggcaggtgccggaggggaggacagggaacagtatttccacttgttctcagggcagcgtgcccgcttccgctgtcctggtgttttactacactccatgtcaggtcagccaactgccctaactgtacatttcacaggctaatgggataaaggactgtgctttaaagataaaaatatcatcatagtaaaagaaatgagggcatatcggctcacaaagagataaactacataggggtgtttatttgtgtcacaaagaatttaaaataacacttgcccatgctatttgttcttcaagaactttctctgccatcaactactattcaaaacctcaaatccacccatatgttaaaattctcattactcttaaggaatagaagcaaattaaacggtaacatccaaaagcaaccacaaacctagtacgacttcattccttccactaactcatagtttgttatatcctagactagacatgcgaaagtttgcctttgtaccatataaagggggagggaaatagctaataatgttaaccaaggaaatatattttaccatacatttaaagttttggccaccacatgtatcacgggtcacttgaaattctttcagctatcagtaggctaatgtcaaaattgtttaaaaattcttgaaagaattttcctgagacaaattttaacttcttgtctatagttgtcagtattattctactatactgtacatgaaagtagcagtgtgaagtacaataattcatattcttcatatccttcttacacgactaagttgaattagtaaagttagattaaataaaacttaaatctcactctaggagttcagtggagaggttagagccagccacacttgaacctaataccctgcccttgacatctggaaacctctacatatttatataacgtgatacatttggataaacaacattgagattatgatgaaaacctacatattccatgtttggaagacccttggaagaggaaaattggattcccttaaacaaaagtgtttaagattgtaattaaaatgatagttgattttcaaaagcattaattttttttcattgtttttaactttgctttcatgaccatcctgccatccttgactttgaactaatgataaagtaatgatctcaaactatgacagaaaagtaatgtaaaatccatccaatctattatttctctaattatgcaattagcctcatagttattatccagaggacccaactgaactgaactaatccttctggcagattcaaatcgtttatttcacacgctgttctaatggcacttatcattagaatcttaccttgtgcagtcatcagaaattccagcgtactataatgaaaacatccttgttttgaaaacctaaaagacaggctctgtatatatatatacttaagaatatgctgacttcacttattagtcttagggatttattttcaattaatattaattttctacaaataattttagtgtcatttccatttggggatattgtcatatcagcacatattttctgtttggaaacacactgttgtttagttaagttttaaataggtgtattacccaagaagtaaagatggaaacgtt

SEQ ID NO: 17 - DNA, 2520 bp, Homo sapiens
cggtggaggccacagacacctcaaacctggattccacaattctacgttaagtgttggagtttttattactctgctgtaggaaagcctttgccaatgcttacaaggaactgtttatccctgcttctctgggttctgtttgatggaggtctcctaacaccactacaaccacagccacagcagactttagccacagagccaagagaaaatgttatccatctgccaggacaacggtcacatttccaacgtgttaaacgtggctgggtatggaatcaattttttgtgctggaagaatacgtgggctccgagcctcagtatgtgggaaagctccattccgacttagacaagggagagggcactgtgaaatacaccctctcaggagatggcgctggcaccgtttttaccattgatgaaaccacaggggacattcatgcaataaggagcctagatagagaagagaaacctttctacactcttcgtgctcaggctgtggacatagaaaccagaaagcccctggagcctgaatcagaattcatcatcaaagtgcaggatattaatgataatgagccaaagtttttggatggaccttatgttgctactgttccagaaatgtctcctgtgggtgcatatgtactccaggtcaaggccacagatgcagatgacccgacctatggaaacagtgccagagtcgtttacagcattcttcagggacaaccttatttctctattgatcccaagacaggtgttattagaacagctttgccaaacatggacagagaagtcaaagaacaatatcaagtactcatccaagccaaggatatgggaggacagcttggaggattagccggaacaacaatagtcaacatcactctcaccgatgtcaatgacaatccacctcgattccccaaaagcatcttccacttgaaagttcctgagtcttcccctattggttcagctattggaagaataagagctgtggatcctgattttggacaaaatgcagaaattgaatacaatattgttccaggagatgggggaaatttgtttgacatcgtcacagatgaggatacacaagagggagtcatcaaattgaaaaagcctttagattttgaaacaaagaaggcatacactttcaaagttgaggcttccaaccttcaccttgaccaccggtttcactcggcgggccctttcaaagacacagctacggtgaagatcagcgtgctggacgtagatgagccaccggttttcagcaagccgctctacaccatggaggtttatgaagacactccggtagggaccatcattggcgctgtcactgctcaagacctggatgtaggcagcggtgctgttaggtacttcatagattggaagagtgatggggacagctactttacaatagatggaaatgaaggaaccatcgccactaatgaattactagacagagaaagcactgcgcagtataatttctccataattgcgagtaaagttagtaaccctttattgaccagcaaagtcaatatactgattaatgtcttagatgtaaatgaatttcctccagaaatatctgtgccatatgagacagccgtgtgtgaaaatgccaagccaggacagataattcagatagtcagtgctgcagaccgagatctttcacctgctgggcaacaattctcctttagattatcacctgaggctgctatcaaaccaaattttacagttcgtgacttcagaaacaacacagcggggattgaaacccgaagaaatggatacagccgcaggcagcaagagttgtatttcctccctgttgtaatagaagacagcagctaccctgtccagagcagcacaaacacaatgactattcgagtctgtagatgtgactctgatggcaccatcctgtcttgtaatgtggaagcaatttttctacctgtaggacttagcactggggcgttgattgcaattctactatgcattgttatactcttagccatagttgtactgtatgtagcactgcgaaggcagaagaaaaagcacaccctgatgacctctaaagaagacatcagagacaacgtcatccattacgatgatgaaggaggtggggaggaagatacccaggctttcgacatcggggctctgagaaacccaaaagtgattgaggagaacaaaattcgcagggatataaaaccagactctctctgtttacctcgtcagagaccacccatggaagataacacagacataagggatttcattcatcaaaggctacaggaaaatgatgtagatccaactgccccaccaatcgattcactggccacatatgcctacgaagggagtgggtccgtggcagagtccctcagctctatagactctctcaccacagaagccgaccaggactatgactatctgacagactggggaccccgctttaaagtcttggcagacatgtttggcgaagaagagagttataaccctgataaagtcacttaagggagtcgtggaggctaaaatacaaccgagaggggagatttt

SEQ ID NO: 18 - DNA, 2545 bp, Homo sapiens
caggaaatgctcttggatctctggactccattaataatattatggattactcttcccccttgcatttacatggctccgatgaatcagtctcaagttttaatgagtggatcccctttggaactaaacagtctgggtgaagaacagcgaattttgaaccgctccaaaagaggctgggtttggaatcaaatgtttgtcctggaagagttttctggacctgaaccgattcttgttggccggctacacacagacctggatcctgggagcaaaaaaatcaagtatatcctatcaggtgatggagctgggaccatatttcaaataaatgatgtaactggagatatccatgctataaaaagacttgaccgggaggaaaaggctgagtataccctaacagctcaagcagtggactgggagacaagcaaacctctggagcctccttctgaatttattattaaagttcaagacatcaatgacaatgcaccagagtttcttaatggaccctatcatgctactgtgccagaaatgtccattttgggtacatctgtcactaacgtcactgcgaccgacgctgatgacccagtttatggaaacagtgcaaagttggtttatagtatattggaagggcagccttatttttccattgagcctgaaacagctattataaaaactgcccttcccaacatggacagagaagccaaggaggagtacctggttgttatccaagccaaagatatgggtggacactctggtggcctgtctgggaccacgacacttacagtgactcttactgatgttaatgacaatcctccaaaatttgcacagagcctgtatcacttctcagtaccggaagatgtggttcttggcactgcaataggaagggtgaaggccaatgatcaggatattggtgaaaatgcacagtcatcatatgatatcatcgatggagatggaacagcactttttgaaatcacttctgatgcccaggcccaggatggcattataaggctaagaaaacctctggactttgagaccaaaaaatcctatacgctaaaggatgaggcagccaatgtccatattgacccacgcttcagtggcagggggccctttaaagacacggcgacagtcaaaatcgtggttgaagatgctgatgagcctccggtcttctcttcaccgacttacctacttgaagttcatgaaaatgctgctctaaactccgtgattgggcaagtgactgctcgtgaccctgatatcacttccagtcctataaggttttccatcgaccggcacactgacctggagaggcagttcaacattaatgcagacgatgggaagataacgctggcaacaccacttgacagagaattaagtgtatggcacaacataacaatcattgctactgaaattaggaaccacagtcagatatcacgagtacctgttgctattaaagtgctggatgtcaatgacaacgcccctgaattcgcatccgaatatgaggcatttttatgtgaaaatggaaaacccggccaagtcattcaaactgttagcgccatggacaaagatgatcccaaaaacggacattatttcttatacagtctccttccagaaatggtcaacaatccgaatttcaccatcaagaaaaatgaagataattccctcagtattttggcaaagcataatggattcaaccgccagaagcaagaagtctatcttttaccaatcataatcagtgatagtggaaatcctccactgagcagcactagcaccttgacaatcagggtctgtggctgcagcaatgacggtgtcgtccagtcttgcaatgtcgaagcttatgtccttccaattggactcagtatgggcgccttaattgccatattagcatgcatcattttgctgttagtcatcgtggtgctgtttgtaactctacggcggcatcaaaaaaatgaaccattaattatcaaagatgatgaagacgttcgagaaaacatcattcgctacgatgatgaaggaggaggggaggaggacacagaggcttttgacattgcaactttacaaaatccagatggaattaatggatttttaccccgtaaggatattaaaccagatttgcagtttatgccaaggcaagggcttgctccagttccaaatggtgttgatgtcgatgaatttataaatgtaaggctgcatgaggcagataatgatcccacagccccgccatatgactccattcaaatatatggctatgaaggccgagggtcagtggctggctccctcagctccttggagtccaccacatcagactcagaccagaattttgactacctcagtgactggggtccccgctttaagagactgggcgaactctactctgttggtgaaagtgacaaagaaacttgacagtggattataaataaatcactggaactgagcattctgtaatattctagggtcactccccttagatacaaccaatgtggctatttgtttagaggcaagtttagcaccagtcatctataactcaaccacatttaatgttgacaaaaagataataaat

SEQ ID NO: 19 - DNA, 2625 bp, Homo sapiens
cggcagccctgacgtgatgagctcaaccagcagagacattccatcccaagagaggtctgcgtgacgcgtccgggaggccaccctcagcaagaccaccgtacagttggtggaaggggtgacagctgcattctcctgtgcctaccacgtaaccaaaaatgaaggagaactactgtttacaagccgccctggtgtgcctgggcatgctgtgccacagccatgcctttgccccagagcggcgggggcacctgcggccctccttccatgggcaccatgagaagggcaaggaggggcaggtgctacagcgctccaagcgtggctgggtctggaaccagttcttcgtgatagaggagtacaccgggcctgaccccgtgcttgtgggcaggcttcattcagatattgactctggtgatgggaacattaaatacattctctcaggggaaggagctggaaccatttttgtgattgatgacaaatcagggaacattcatgccaccaagacgttggatcgagaagagagagcccagtacacgttgatggctcaggcggtggacagggacaccaatcggccactggagccaccgtcggaattcattgtcaaggtccaggacattaatgacaaccctccggagttcctgcacgagacctatcatgccaacgtgcctgagaggtccaatgtgggaacgtcagtaatccaggtgacagcttcagatgcagatgaccccacttatggaaatagcgccaagttagtgtacagtatcctcgaaggacaaccctatttttcggtggaagcacagacaggtatcatcagaacagccctacccaacatggacagggaggccaaggaggagtaccacgtggtgatccaggccaaggacatgggtggacatatgggcggactctcagggacaaccaaagtgacgatcacactgaccgatgtcaatgacaacccaccaaagtttccgcagaggctataccagatgtctgtgtcagaagcagccgtccctggggaggaagtaggaagagtgaaagctaaagatccagacattggagaaaatggcttagtcacatacaatattgttgatggagatggtatggaatcgtttgaaatcacaacggactatgaaacacaggagggggtgataaagctgaaaaagcctgtagattttgaaaccgaaagagcctatagcttgaaggtagaggcagccaacgtgcacatcgacccgaagtttatcagcaatggccctttcaaggacactgtgaccgtcaagatctcagtagaagatgctgatgagccccctatgttcttggccccaagttacatccacgaagtccaagaaaatgcagctgctggcaccgtggttgggagagtgcatgccaaagaccctgatgctgccaacagcccgataaggtattccatcgatcgtcacactgacctcgacagatttttcactattaatccagaggatggttttattaaaactacaaaacctctggatagagaggaaacagcctggctcaacatcactgtctttgcagcagaaatccacaatcggcatcaggaagcccaagtcccagtggccattagggtccttgatgtcaacgataatgctcccaagtttgctgccccttatgaaggtttcatctgtgagagtgatcagaccaagccactttccaaccagccaattgttacaattagtgcagatgacaaggatgacacggccaatggaccaagatttatcttcagcctaccccctgaaatcattcacaatccaaatttcacagtcagagacaaccgagataacacagcaggcgtgtacgcccggcgtggagggttcagtcggcagaagcaggacttgtaccttctgcccatagtgatcagcgatggcggcatcccgcccatgagtagcaccaacaccctcaccatcaaagtctgcgggtgcgacgtgaacggggcactgctctcctgcaacgcagaggcctacattctgaacgccggcctgagcacaggcgccctgatcgccatcctcgcctgcatcgtcattctcctggtcattgtagtattgtttgtgaccctgagaaggcaaaagaaagaaccactcattgtctttgaggaagaagatgtccgtgagaacatcattacttatgatgatgaagggggtggggaagaagacacagaagcctttgatattgccaccctccagaatcctgatggtatcaatggatttatcccccgcaaagacatcaaacctgagtatcagtacatgcctagacctgggctccggccagcgcccaacagcgtggatgtcgatgacttcatcaacacgagaatacaggaggcagacaatgaccccacggctcctccttatgactccattcaaatctacggttatgaaggcaggggctcagtggccgggtccctgagctccctagagtcggccaccacagattcagacttggactatgattatctacagaactggggacctcgttttaagaaactagcagatttgtatggttccaaagacacttttgatgacgattcttaacaataacgatacaaatttggccttaagaactgtgtctggcgttctcaagaatctagaagatgtgtaacaggtatttttt

SEQ ID NO: 20 - DNA, 4098 bp, Homo sapiens
gacggtcggctgacaggctccacagagctccactcacgctcaggccctggacggacaggcagtccaacggaacagaaacatccctcagccccacaggcacgatctgttcctcctgggaagatgcagaggctcatgatgctcctcgccacatcgggcgcctgcctgggcctgctggcagtggcagcagtggcagcagcaggtgctaaccctgcccaacgggacacccacagcctgctgcccacccaccggcgccaaaagagagattggatttggaaccagatgcacattgatgaagagaaaaacacctcacttccccatcatgtaggcaagatcaagtcaagcgtgagtcgcaagaatgccaagtacctgctcaaaggagaatatgtgggcaaggtcttccgggtcgatgcagagacaggagacgtgttcgccattgagaggctggaccgggagaatatctcagagtaccacctcactgctgtcattgtggacaaggacactggtgaaaacctggagactccttccagcttcaccatcaaagttcatgacgtgaacgacaactggcctgtgttcacgcatcggttgttcaatgcgtccgtgcctgagtcgtcggctgtggggacctcagtcatctctgtgacagcagtggatgcagacgaccccactgtgggagaccacgcctctgtcatgtaccaaatcctgaaggggaaagagtattttgccatcgataattctggacgtattatcacaataacgaaaagcttggaccgagagaagcaggccaggtatgagatcgtggtggaagcgcgagatgcccagggcctccggggggactcgggcacggccaccgtgctggtcactctgcaagacatcaatgacaacttccccttcttcacccagaccaagtacacatttgtcgtgcctgaagacacccgtgtgggcacctctgtgggctctctgtttgttgaggacccagatgagccccagaaccggatgaccaagtacagcatcttgcggggcgactaccaggacgctttcaccattgagacaaaccccgcccacaacgagggcatcatcaagcccatgaagcctctggattatgaatacatccagcaatacagcttcatcgtcgaggccacagaccccaccatcgacctccgatacatgagccctcccgcgggaaacagagcccaggtcattatcaacatcacagatgtggacgagccccccattttccagcagcctttctaccacttccagctgaaggaaaaccagaagaagcctctgattggcacagtgctggccatggaccctgatgcggctaggcatagcattggatactccatccgcaggaccagtgacaagggccagttcttccgagtcacaaaaaagggggacatttacaatgagaaagaactggacagagaagtctacccctggtataacctgactgtggaggccaaagaactggattccactggaacccccacaggaaaagaatccattgtgcaagtccacattgaagttttggatgagaatgacaatgccccggagtttgccaagccctaccagcccaaagtgtgtgagaacgctgtccatggccagctggtcctgcagatctccgcaatagacaaggacataacaccacgaaacgtgaagttcaaattcaccttgaatactgagaacaactttaccctcacggataatcacgataacacggccaacatcacagtcaagtatgggcagtttgaccgggagcataccaaggtccacttcctacccgtggtcatctcagacaatgggatgccaagtcgcacgggcaccagcacgctgaccgtggccgtgtgcaagtgcaacgagcagggcgagttcaccttctgcgaggatatggccgcccaggtgggcgtgagcatccaggcagtggtagccatcttactctgcatcctcaccatcacagtgatcaccctgctcatcttcctgcggcggcggctccggaagcaggcccgcgcgcacggcaagagcgtgccggagatccacgagcagctggtcacctacgacgaggagggcggcggcgagatggacaccaccagctacgatgtgtcggtgctcaactcggtgcgccgcggcggggccaagcccccgcggcccgcgctggacgcccggccttccctctatgcgcaggtgcagaagccaccgaggcacgcgcctggggcacacggagggcccggggagatggcagccatgatcgaggtgaagaaggacgaggcggaccacgacggcgacggccccccctacgacacgctgcacatctacggctacgagggctccgagtccatagccgagtccctcagctccctgggcaccgactcatccgactctgacgtggattacgacttccttaacgactggggacccaggtttaagatgctggctgagctgtacggctcggacccccgggaggagctgctgtattaggcggccgaggtcactctgggcctggggacccaaaccccctgcagcccaggccagtcagacgccaggcaccacagcctccaaaaatggcagtgactccccagcccagcaccccttcctcgtgggtcccagagacctcatcagccttgggatagcaaactccaggttcctgaaatatccaggaatatatgtcagtgatgactattctcaaatgctggcaaatccaggctggtgttctgtctgggctcagacatccacataaccctgtcacccacagaccgccgtctaactcaaagacttcctctggctccccaaggctgcaaagcaaaacagactgtgtttaactgctgcagggtctttttctagggtccctgaacgccctggtaaggctggtgaggtcctggtgcctatctgcctggaggcaaaggcctggacagcttgacttgtggggcaggattctctgcagcccattcccaagggagactgaccatcatgccctctctcgggagccctagccctgctccaactccatactccactccaagtgccccaccactccccaacccctctccaggcctgtcaagagggaggaaggggccccatggcagctcctgaccttgggtcctgaagtgacctcactggcctgccatgccagtaactgtgctgtactgagcactgaaccacattcagggaaatggcttattaaactttgaagcaactgtgaattcattctggaggggcagtggagatcaggagtgacagatcacagggtgagggccacctccacacccaccccctctggagaaggcctggaagagctgagaccttgctttgagactcctcagcacccctccagttttgcctgagaaggggcagatgttcccggagcagaagacgtctccccttctctgcctcacctggtcgccaatccatgctctctttcttttctctgtctactccttatcccttggtttagaggaacccaagatgtggcctttagcaaaactggacaatgtccaaacccactcatgactgcatgacggagccgagccatgtgtctttacacctcgctgttgtcacatctcagggaactgaccctcaggcacaccttgcagaaggcaaggccctgccctgcccaacctctgtggtcacccatgcatcttccactggaacgtttcactgcaaacacaccttggagaagtggcatcagtcaacagagaggggcagggaaggagacaccaagctcacccttcgtcatggaccgaggttcccactctgggcaaagcccctcacactgcaagggattgtagataacactgacttgtttgttttaaccaataactagcttcttataatgatttttttactaatgatacttacaagtttctagctctcacagacatatagaataagggtttttgcataataagcaggttgttatttaggttaacaatattaattcaggttttttagttggaaaaacaattcctgtaaccttctattttctataattgtagtaattgctctacagataatgtctatatattggccaaactggtgcatgacaagtactgtatttttttatacctaaataaagaaaaatctttagcctgggcaacaaaaaaa

SEQ ID NO: 21 - DNA, 21 bp, Artificial sequence
  Synthetic oligonucleotide
gagaggtccacgagggagccc

SEQ ID NO: 22 - DNA, 20 bp, Artificial sequence
  Synthetic oligonucleotide
cacggctcggaggccgcgca

SEQ ID NO: 23 - DNA, 20 bp, Artificial sequence
  Synthetic oligonucleotide
cgcctccaaggtcacttcag

SEQ ID NO: 24 - DNA, 20 bp, Artificial sequence
  Synthetic oligonucleotide
cgcctccaaggtcacttcag

SEQ ID NO: 25 - DNA, 20 bp, Artificial sequence
  Synthetic oligonucleotide
agtgaccttctttcctggac

SEQ ID NO: 26 - DNA, 20 bp, Artificial sequence
  Synthetic oligonucleotide
gtttggatgggaagatcttc

SEQ ID NO: 27 - DNA, 20 bp, Artificial sequence
  Synthetic oligonucleotide
cttgtgtcttcgtaagatac

SEQ ID NO: 28 - DNA, 20 bp, Artificial sequence
  Synthetic oligonucleotide
ctgggggaagggacccttgc

SEQ ID NO: 29 - DNA, 20 bp, Artificial sequence
  Synthetic oligonucleotide
cttcagcacaaaaggggcct

SEQ ID NO: 30 - DNA, 22 bp, Artificial sequence
  Synthetic oligonucleotide
caacgactttggagggtgggac

SEQ ID NO: 31 - DNA, 21 bp, Artificial sequence
  Synthetic oligonucleotide
gttgttcctcacaaactgctc

SEQ ID NO: 32 - DNA, 21 bp, Artificial sequence
  Synthetic oligonucleotide
gtggtgggagggcttccattg

SEQ ID NO: 33 - DNA, 21 bp, Artificial sequence
  Synthetic oligonucleotide
gatctgacggggctcagggac

SEQ ID NO: 34 - DNA, 20 bp, Artificial sequence
  Synthetic oligonucleotide
catctgtgagctgggcctgg

SEQ ID NO: 35 - DNA, 21 bp, Artificial sequence
  Synthetic oligonucleotide
ccttcctcgttgacctctgcc

SEQ ID NO: 36 - DNA, 22 bp, Artificial sequence
  Synthetic oligonucleotide
ctttgttgccatggtcagacag

SEQ ID NO: 37 - DNA, 20 bp, Artificial sequence
  Synthetic oligonucleotide
gcagcaccagcaggaggaac

SEQ ID NO: 38 - DNA, 21 bp, Artificial sequence
  Synthetic oligonucleotide
ggttggtgccacgtcattgcg

SEQ ID NO: 39 - DNA, 22 bp, Artificial sequence
  Synthetic oligonucleotide
gttggctggccgaggacggtac

SEQ ID NO: 40 - protein, 10 aa, Artificial sequence
  Synthetic peptide
VPENGKGPFP

SEQ ID NO: 41 - protein, 19 aa, Artificial sequence
  Synthetic peptide
QEPKDPHDLMFTIHRSTGT

SEQ ID NO: 42 - protein, 10 aa, Artificial sequence
  Synthetic peptide
DNGSPPTTGT

SEQ ID NO: 43 - protein, 23 aa, Artificial sequence
  Synthetic peptide
TDKDLSPHTSPFQAQLTDDSDIY

SEQ ID NO: 44 - protein, 16 aa, Artificial sequence
  Synthetic peptide
DCHGHVETCPGPWKGG

SEQ ID NO: 45 - protein, 12 aa, Artificial sequence
  Synthetic peptide
MYRPRPANPDEI

SEQ ID NO: 46 - DNA, 19 bp, Artificial sequence
  Synthetic oligonucleotide
cttggagatgctctgtggc

SEQ ID NO: 47 - DNA, 20 bp, Artificial sequence
  Synthetic oligonucleotide
gcacttgctgtctgctggtc

SEQ ID NO: 48 - DNA, 20 bp, Artificial sequence
  Synthetic oligonucleotide
catgcttgttctcctgtgtg

SEQ ID NO: 49 - DNA, 20 bp, Artificial sequence
  Synthetic oligonucleotide
ctgtgacatcatctgtcttg

SEQ ID NO: 50 - DNA, 22 bp, Artificial sequence
  Synthetic oligonucleotide
caaagagactacagcaatggac

SEQ ID NO: 51 - DNA, 20 bp, Artificial sequence
  Synthetic oligonucleotide
ctgagtgaggacatctgcag

SEQ ID NO: 52 - DNA, 19 bp, Artificial sequence
  Synthetic oligonucleotide
ctgggtgacagagtgagac

SEQ ID NO: 53 - DNA, 20 bp, Artificial sequence
  Synthetic oligonucleotide
cttcatggtgtactcagatc

SEQ ID NO: 54 - DNA, 22 bp, Artificial sequence
  Synthetic oligonucleotide
ggttctagaggagatcattgtc

SEQ ID NO: 55 - DNA, 20 bp, Artificial sequence
  Synthetic oligonucleotide
gtcttgagaggtgagagctg

SEQ ID NO: 56 - DNA, 20 bp, Artificial sequence
  Synthetic oligonucleotide
gcatgagccactgcatccag

SEQ ID NO: 57 - DNA, 20 bp, Artificial sequence
  Synthetic oligonucleotide
gccctgaatgatgacatcag

SEQ ID NO: 58 - DNA, 22 bp, Artificial sequence
  Synthetic oligonucleotide
caatctctatggtaatcagaac

SEQ ID NO: 59 - DNA, 21 bp, Artificial sequence
  Synthetic oligonucleotide
catctcaactgtcctgcacag

SEQ ID NO: 60 - DNA, 22 bp, Artificial sequence
  Synthetic oligonucleotide
cagtgactcttacctatttatg

SEQ ID NO: 61 - DNA, 20 bp, Artificial sequence
  Synthetic oligonucleotide
catcctgccgctgtgtatac

SEQ ID NO: 62 - DNA, 20 bp, Artificial sequence
  Synthetic oligonucleotide
cagccatagtgctgagactg

SEQ ID NO: 63 - DNA, 20 bp, Artificial sequence
  Synthetic oligonucleotide
cacccatgagccagtgcttc

SEQ ID NO: 64 - DNA, 20 bp, Artificial sequence
  Synthetic oligonucleotide
gcttctgctctcagagtcag

SEQ ID NO: 65 - DNA, 19 bp, Artificial sequence
  Synthetic oligonucleotide
gtagacagggctggagttg

SEQ ID NO: 66 - DNA, 20 bp, Artificial sequence
  Synthetic oligonucleotide
cagagctctgctctaggatc

SEQ ID NO: 67 - DNA, 21 bp, Artificial sequence
  Synthetic oligonucleotide
ctgttcagtgagcagattctc

SEQ ID NO: 68 - DNA, 21 bp, Artificial sequence
  Synthetic oligonucleotide
cagtagcaagaaatctcatgc

SEQ ID NO: 69 - DNA, 21 bp, Artificial sequence
  Synthetic oligonucleotide
caataggctcatctaggtctc

SEQ ID NO: 70 - DNA, 20 bp, Artificial sequence
  Synthetic oligonucleotide
gactaacactacctcctctg

SEQ ID NO: 71 - DNA, 20 bp, Artificial sequence
  Synthetic oligonucleotide
gtccatgaatgtctatgatc

SEQ ID NO: 72 - DNA, 21 bp, Artificial sequence
  Synthetic oligonucleotide
gatgtcataggcgctctgctg

SEQ ID NO: 73 - DNA, 19 bp, Artificial sequence
  Synthetic oligonucleotide
gtcgcggcagctgcttcac

SEQ ID NO: 74 - DNA, 20 bp, Artificial sequence
  Synthetic oligonucleotide
gcagagagtgaaggaggctg

SEQ ID NO: 75 - DNA, 20 bp, Artificial sequence
  Synthetic oligonucleotide
gtactgaggaggctgaggag